(19) United States
(12) Patent Application Publication
(10) Pub. No.: US 2023/0263810 A1
(43) Pub. Date: Aug. 24, 2023

(54) SYSTEMS, METHODS, AND COMPOSITIONS FOR INFECTIONS

(71) Applicant: Ojai Energetics PBC, Ojai, CA (US)

(72) Inventor: William KLEIDON, Ojai, CA (US)

(21) Appl. No.: 17/951,981

(22) Filed: Sep. 23, 2022

Related U.S. Application Data
(63) Continuation of application No. PCT/US2021/024710, filed on Mar. 29, 2021.
(60) Provisional application No. 63/002,162, filed on Mar. 30, 2020.
(60) Provisional application No. 63/005,061, filed on Apr. 3, 2020.

(51) Int. Cl. A61K 31/00; A61K 45/06; A61K 9/50; A61K 9/00; A61P 31/16

(52) U.S. Cl. CPC A61K 31/658; A61K 45/06; A61K 9/5084; A61K 9/0053; A61P 31/16

(57) ABSTRACT

The present disclosure provides compositions, systems, and methods for treating

### CROSS-REFERENCE

This application is a continuation of International Patent Application No. PCT/US2021/024710, filed on Mar. 29, 2021, claims the benefit of U.S. Provisional Application No. 63/002,162, filed Mar. 30, 2020, and U.S. Provisional Application No. 63/005,061, filed Apr. 3, 2020, each of which is entirely incorporated herein by reference.

### BACKGROUND

Viral infections may be particularly difficult to treat, for example, because of their evasiveness against natural and induced immune responses. Therapeutic compositions can suffer from drawbacks limiting their utility for administration to subjects. For example, oral compositions can be destroyed, broken down, or cleared by the liver of a subject, resulting in reduced delivery to the subject. In some cases, therapeutic compositions can have low bioavailability and shelf life stability.

### SUMMARY

There remains a substantial need for compositions having increased bioavailability, increased shelf stability, reduced first pass metabolism, and/or other beneficial properties. There remains a substantial need for methods and compositions comprising one or more therapeutic moieties (e.g., a cannabinoid compound and/or a non-cannabinoid compound) for administering to a subject who may have or may be suspected of having a viral infection.

Provided herein are systems, methods, and compositions for treatment of infections, such as virus infections.

In an aspect, provided herein is a method comprising administering to a subject in need thereof a therapeutically effective amount of a composition comprising a plurality of microcapsules, a microcapsule of the plurality comprising at least one cannabinoid compound, wherein the administering effects, in the subject, one or more members selected from the group consisting of (i) reduced expression or activity of a cytokine in a target cell, when the administering occurs to the subject having a viral infection, (ii) reduced platelet activation, and (iii) reduced expression or activity of angiotensin pathway protein in a target cell, as compared to a corresponding control.

In some embodiments, the administering effects the reduced expression or activity of the cytokine in the target cell.

In some embodiments of any one of the methods disclosed herein, the administering effects at least about 10%, 20%, 30%, 40%, 50%, 60%, or more reduction in the expression or activity of (i) the cytokine and/or (ii) an additional cytokine in the target cell. In some embodiments of any one of the methods disclosed herein, the cytokine or the additional cytokine is selected from the group consisting of an interferon, an interleukin, a chemokine, a colony-stimulating factor, and a tumor necrosis factor. In some embodiments of any one of the methods disclosed herein, the administering effects reduced cytokine storm in the subject.

In some embodiments of any one of the methods disclosed herein, the administering effects the reduced platelet activation. In some embodiments of any one of the methods disclosed herein, the administering effects at least about 10%, 20%, 30%, 40%, 50%, 60%, or more reduction in expression level of a chemokine in the platelet, wherein the chemokine is selected from the group consisting of CXCL1, CXCL4, CXCLS, CXCL7, CXCL8, CXCL12, CCL3, and CCLS. In some embodiments of any one of the methods disclosed herein, the administering effects at least about 10%, 20%, 30%, 40%, 50%, 60%, or more reduction in expression level of a cell-adhesion molecule in the platelet, wherein the cell-adhesion molecule is selected from the group consisting of glycoprotein P-selectin, and integrin. In some embodiments of any one of the methods disclosed herein, the administering effects at least about 10%, 20%, 30%, 40%, 50%, 60%, or more reduction in expression level of adenosine diphosphate (ADP) and/or adenosine triphosphate (ATP) in the platelet. In some embodiments of any one of the methods disclosed herein, the administering effects reduced blood clot formation in (i) a blood vessel of the subject and/or (ii) a blood sample derived from the subject. In some embodiments of any one of the methods disclosed herein, upon the administering, the blood sample exhibits at least about 10%, 20%, 30%, 40%, 50%, 60%, or more reduction in platelet aggregation as measured by light-transmission aggregometry (LTA) assay and/or whole blood aggregometry (WBA) assay.

In some embodiments of any one of the methods disclosed herein, the administering effects the reduced expression or activity of the angiotensin pathway protein in the target cell. In some embodiments of any one of the methods disclosed herein, the administering effects at least about 10%, 20%, 30%, 40%, 50%, 60%, or more reduction in the expression or activity of the angiotensin pathway protein. In some embodiments of any one of the methods disclosed herein, the angiotensin pathway protein is selected from the group consisting of angiotensin converting enzyme (ACE) and angiotensin receptor. In some embodiments of any one of the methods disclosed herein, the angiotensin pathway protein is selected from the group consisting of ACE2 and angiotensin II receptor.

In some embodiments of any one of the methods disclosed herein, the method comprises administering to the subject (i) the composition further comprising an additional therapeutic compound and/or (ii) an additional composition comprising the additional therapeutic compound, wherein the additional therapeutic compound is selected from the group consisting of hydroxychloroquine, chroloquine, azithromycin, dexamethasone, steroids, vitamins C, vitamin D, stem cells, baloxavir, chloroquine phosphate, lopinavir, ritonavir, neuraminidase inhibitors, remedesivir, ascorbic acid, methylprednisolone, nitric oxide, sarilumab, sirolimus, tocilizumab, ACE inhibitors, angiotensin II receptor blockers (ARBs), ibuprofen, indomethacin, and niclosamide.

In some embodiments of any one of the methods disclosed herein, the additional therapeutic compound is encapsulated in a microcapsule of the plurality of microcapsules. In some embodiments of any one of the methods disclosed herein, the at least one cannabinoid compound and the additional therapeutic compound are encapsulated in the same microcapsule. In some embodiments of any one of the methods disclosed herein, the at least one cannabinoid compound and the additional therapeutic compound are encapsulated in different microcapsules.

In some embodiments of any one of the methods disclosed herein, the additional therapeutic compound is provided in non-encapsulated form

In some embodiments of any one of the methods disclosed herein, the subject has or is suspected of having a viral infection (or infection of a virus). In some embodiments of any one of the methods disclosed herein, the viral infection is from a coronavirus. In some embodiments of any one of the methods disclosed herein, the administering effects, in the subject, reduced viral infection in a target cell, as compared to a corresponding control. In some embodiments of any one of the methods disclosed herein, the administering effects, in the subject, reduced replication of the virus in the target cell, as compared to a corresponding control. In some embodiments of any one of the methods disclosed herein, the target cell is an epithelial cell.

In some embodiments of any one of the methods disclosed herein, the corresponding control is a control composition comprising the at least one cannabinoid compound in non-encapsulated form. In some embodiments of any one of the methods disclosed herein, the corresponding control is the subject prior to the administering.

In another aspect, provided herein is a composition comprising at least one cannabinoid compound for use in any one of the methods disclosed herein. In some embodiments, the composition is for use in preventing or treating a condition of a subject. In some embodiments, the condition is a viral infection.

In another aspect, provided is a composition for use in preventing or treating a viral infection, the composition comprising a plurality of microcapsules, wherein a microcapsule of the plurality comprises at least one cannabinoid compound, and wherein administering the composition to a subject in need thereof effects, in the subject, one or more members selected from the group consisting of (i) reduced expression or activity of a cytokine in a target cell, when the administering occurs to the subject having a viral infection, (ii) reduced platelet activation, and (iii) reduced expression or activity of angiotensin pathway protein in a target cell, as compared to a corresponding control.

In some embodiments, the administering effects the reduced expression or activity of the cytokine in the target cell.

In some embodiments of any one of the compositions disclosed herein, the administering effects at least about 10%, 20%, 30%, 40%, 50%, 60%, or more reduction in the expression or activity of (i) the cytokine and/or (ii) an additional cytokine in the target cell. In some embodiments of any one of the compositions disclosed herein, the cytokine or the additional cytokine is selected from the group consisting of an interferon, an interleukin, a chemokine, a colony-stimulating factor, and a tumor necrosis factor. In some embodiments of any one of the compositions disclosed herein, the administering effects reduced cytokine storm in the subject.

In some embodiments of any one of the compositions disclosed herein, the administering effects the reduced platelet activation. In some embodiments of any one of the compositions disclosed herein, the administering effects at least about 10%, 20%, 30%, 40%, 50%, 60%, or more reduction in expression level of a chemokine in the platelet, wherein the chemokine is selected from the group consisting of CXCL1, CXCL4, CXCLS, CXCL7, CXCL8, CXCL12, CCL3, and CCLS. In some embodiments of any one of the compositions disclosed herein, the administering effects at least about 10%, 20%, 30%, 40%, 50%, 60%, or more reduction in expression level of a cell-adhesion molecule in the platelet, wherein the cell-adhesion molecule is selected from the group consisting of glycoprotein P-selectin, and integrin. In some embodiments of any one of the compositions disclosed herein, the administering effects at least about 10%, 20%, 30%, 40%, 50%, 60%, or more reduction in expression level of adenosine diphosphate (ADP) and/or adenosine triphosphate (ATP) in the platelet. In some embodiments of any one of the compositions disclosed herein, the administering effects reduced blood clot formation in (i) a blood vessel of the subject and/or (ii) a blood sample derived from the subject. In some embodiments of any one of the compositions disclosed herein, upon the administering, the blood sample exhibits at least about 10%, 20%, 30%, 40%, 50%, 60%, or more reduction in platelet aggregation as measured by light-transmission aggregometry (LTA) assay and/or whole blood aggregometry (WBA) assay.

In some embodiments of any one of the compositions disclosed herein, the administering effects the reduced expression or activity of the angiotensin pathway protein in the target cell. In some embodiments of any one of the compositions disclosed herein, the administering effects at least about 10%, 20%, 30%, 40%, 50%, 60%, or more reduction in the expression or activity of the angiotensin pathway protein. In some embodiments of any one of the compositions disclosed herein, the angiotensin pathway protein is selected from the group consisting of angiotensin converting enzyme (ACE) and angiotensin receptor. In some embodiments of any one of the compositions disclosed herein, the angiotensin pathway protein is selected from the group consisting of ACE2 and angiotensin II receptor.

In some embodiments of any one of the compositions disclosed herein, (i) the composition further comprises an additional therapeutic compound and/or (ii) an additional composition for use in preventing or treating a viral infection in the subject comprises the additional therapeutic compound, wherein the additional therapeutic compound is selected from the group consisting of hydroxychloroquine, chroloquine, azithromycin, dexamethasone, steroids, vitamins C, vitamin D, stem cells, baloxavir, chloroquine phosphate, lopinavir, ritonavir, neuraminidase inhibitors, remedesivir, ascorbic acid, methylprednisolone, nitric oxide, sarilumab, sirolimus, tocilizumab, ACE inhibitors, angiotensin II receptor blockers (ARBs), ibuprofen, indomethacin, and niclosamide.

In some embodiments of any one of the compositions disclosed herein, the additional therapeutic compound is encapsulated in a microcapsule of the plurality of microcapsules. In some embodiments of any one of the compositions disclosed herein, the at least one cannabinoid compound and the additional therapeutic compound are encapsulated in the same microcapsule. In some embodiments of any one of the compositions disclosed herein, the at least one cannabinoid compound and the additional therapeutic compound are encapsulated in different microcapsules.

In some embodiments of any one of the compositions disclosed herein, the additional therapeutic compound is provided in non-encapsulated form

In some embodiments of any one of the compositions disclosed herein, the subject has or is suspected of having a viral infection (or infection of a virus). In some embodiments of any one of the compositions disclosed herein, the viral infection is from a coronavirus. In some embodiments of any one of the methods disclosed herein, the administering effects, in the subject, reduced viral infection in a target cell, as compared to a corresponding control. In some embodiments of any one of the compositions disclosed herein, the administering the composition to the subject in need thereof effects, in the subject, reduced replication of the virus in the target cell, as compared to a corresponding control. In some embodiments of any one of the compositions disclosed herein, the target cell is an epithelial cell.

In some embodiments of any one of the compositions disclosed herein, the corresponding control is a control composition comprising the at least one cannabinoid compound in non-encapsulated form. In some embodiments of any one of the compositions disclosed herein, the corresponding control is the subject prior to the administering.

In another aspect, provided herein is a composition comprising: a cannabinoid compound and one or more therapeutic compounds selected from the group consisting of: hydroxychloroquine, chroloquine, azithromycin, dexamethasone, steroids, vitamins C, vitamin D, stem cells, baloxavir, chloroquine phosphate, lopinavir, ritonavir, neuraminidase inhibitors, remedesivir, ascorbic acid, methylprednisolone, nitric oxide, sarilumab, sirolimus, tocilizumab, angiotensin converting enzyme (ACE) inhibitors, angiotensin II receptor blockers (ARBs), ibuprofen, indomethacin, and niclosamide.

In some embodiments, the cannabinoid compound and the one or more therapeutic compounds are provided in a plurality of microcapsules.

In some embodiments, the cannabinoid compound and the one or more therapeutic compounds are provided in non-encapsulated form.

In another aspect, provided herein is a method, comprising: administering the composition to a subject having or suspected of having a disease caused by a coronavirus.

In another aspect, provided herein is a kit comprising any one of the compositions disclosed herein. In another aspect, provided herein is a system comprising any one of the compositions disclosed herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings or figures (also “FIG.” and “FIGs.” herein), of which:

FIG. 1A shows an example of a droplet generator.

FIG. 1B shows another example of a droplet generator.

FIG. 1C shows an example of a microfluidic structure.

FIG. 2A shows an exemplary microscope image of an unprocessed composition of quillaja extract, hemp oil, and water at 400× magnification;

FIG. 2B shows an exemplary microscope image of an unprocessed composition of quillaja extract, hemp oil, and water at 1000× magnification;

FIG. 3A shows an exemplary microscope image of a microfluidic processed composition of quillaja extract, hemp oil, and water at 400× agnification;

FIG. 3B shows an exemplary microscope image of a microfluidic processed composition of quillaja extract, hemp oil, and water at 400× magnification;

FIG. 3C shows an exemplary microscope image of a microfluidic processed composition of quillaja extract, hemp oil, and water at 1000× magnification;

FIG. 3D shows an exemplary microscope image of a microfluidic processed composition of quillaja extract, hemp oil, and water at 1000× magnification;

FIG. 4A shows an exemplary microscope image of a microfluidic processed composition of quillaja extract, hemp oil, water, and sodium alginate at 1000× magnification;

FIG. 4B shows an exemplary microscope image of a microfluidic processed composition of quillaja extract, hemp oil, water, and sodium alginate at 1000× magnification;

FIG. 4C shows an exemplary microscope image of a microfluidic processed composition of quillaja extract, hemp oil, water, and sodium alginate at 1000× magnification; and

FIGS. 5A-5C show examples of therapeutic compositions as disclosed herein.

### DETAILED DESCRIPTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The term “about” or “nearly” as used herein refers to within +/- 10%, 9%, 8%, 7%, 6%, 5%, 4%, 3%, 2%, or 1% of the designated amount.

The present disclosure provides therapeutic compositions, systems and methods for generating such compositions, and methods for use and administration thereof, to subjects affected by, or suspected of affection by, a disease caused by a virus. The present disclosure provides therapeutic compositions, systems and methods for generating such compositions, and methods for use and administration thereof, to subjects affected by, or suspected of affection by, infections caused by a virus.

Examples of a virus can include one or more of the following: (1) a double stranded DNA virus that is enveloped (e.g., Poxviridae, Chordopoxvirinae, Herpesviridae (i.e., Herpes), and/or Hepadnaviridae); (2) a double stranded DNA virus that is nonenveloped (e.g., Adenoviridae, and/or Papovaviridae); (3) a single stranded DNA virus that is nonenveloped (e.g., Parvoviridae); (4) a single stranded RNA virus (e.g., Coronaviridae, Toroviridae, Paramyxoviridae, Bunyaviridae, Orthomyxoviridae (e.g., influenza virus), Arenaviridae, Togaviridae, Flaviviridae, Retroviridae (e.g., human immunodeficiency virus (HIV)), Rhabdoviridae, and/or Filoviridae); (5) a double stranded RNA virus (e.g., Reoviridae and/or Birnaviridae); and/or (6) a single stranded RNA virus (e.g., Picornaviridae and/or Caliciviridae).

The virus can be a coronavirus or an influenza virus. In some cases, the coronavirus can be selected from the group: alphacoronavirus, betacoronavirus, deltacoronavirus, and gammacoronavirus. Examples of alphacoronavirus can include, but are not limited to, Bat coronavirus CDPHE15, Bat coronavirus HKU10, Human coronavirus 229E, Human coronavirus NL63, Miniopterus bat coronavirus 1, Miniopterus bat coronavirus HKU8, Mink coronavirus 1, Porcine epidemic diarrhoea virus, Rhinolophus bat coronavirus HKU2, and Scotophilus bat coronavirus 512. Examples of betacoronavirus can include, but are not limited to, Betacoronavirus 1, Hedgehog coronavirus 1, Human coronavirus HKU1, Middle East respiratory syndrome-related coronavirus, Murine coronavirus, Pipistrellus bat coronavirus HKUS, Rousettus bat coronavirus HKU9, Severe acute respiratory syndrome-related coronavirus, Tylonycteris bat coronavirus HKU4. Examples of deltacoronavirus can include, but are not limited to, Bulbul coronavirus HKU11, Common moorhen coronavirus HKU21, Coronavirus HKU15, Munia coronavirus HKU13, Night heron coronavirus HKU19, Thrush coronavirus HKU12, White-eye coronavirus HKU16, Wigeon coronavirus HKU20. Examples of gammacoronavirus can include, but are not limited to, Avian coronavirus, Beluga whale coronavirus SW1. Additional examples of coronavirus can include MERS-CoV, SARS-CoV, and SARS-Cov-2 (i.e., SARS-COV-2). The virus can be any mutation of the listed examples.

In some cases, the influenza virus can be selected from the genera: Influenza virus A, Influenza virus B, Influenza virus C and Influenza virus D. In further embodiments, the influenza A virus is of the subtype HIN1, H1N2, H2N2, H3N2, H5N1, H6N1, H7N7, H7N2, H7N3, H7N9, H9N2, or H1ON7. The virus can be any mutation of the listed examples.

In some cases, a virus can infect a cell (e.g., a cell of a subject). In some embodiments, the cell is a somatic cell. In some embodiments, the cell is a stem cell or a progenitor cell. In some embodiments, the cell is a mesenchymal stem or progenitor cell. In some embodiments, the cell is a hematopoietic stem or progenitor cell. In some embodiments, the cell is a muscle cell, a skin cell, a blood cell, or an immune cell. Other exemplary cells can include lymphoid cells, such as B cell, T cell (Cytotoxic T cell, Natural Killer T cell, Regulatory T cell, T helper cell), Natural killer cell, cytokine induced killer (CIK) cells; myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

This disclosure provides for encapsulation of therapeutic compositions, and methods for the manufacture, delivery, and use of such compositions. Therapeutic compositions can be encapsulated, including in microcapsules. Microencapsulation can provide benefits such as shelf stability, improved bioavailability, reduced first-pass metabolism, and extended or modified release profiles. Microencapsulation may involve generating a plurality of droplets in an emulsion. Microencapsulation can increase solubility of the therapeutic compositions in water (e.g., solubility of oil-based therapeutic compositions), such as to ease delivery and/or administration of the therapeutic compositions to a subject. The therapeutic compositions of the present disclosure can comprise cannabinoids, terpenes, essential oils, and other desirable compounds.

Any of the therapeutic compositions provided herein may be administered in non-encapsulated form.

A therapeutic composition may comprise a cannabinoid compound. Examples of cannabinoid compounds are described elsewhere herein.

A therapeutic composition may comprise a therapeutic composition (e.g., a non-cannabinoid therapeutic composition or compound, as used interchangeably herein). One or more therapeutic compounds may be selected from the group of, in any permutation: hydroxychloroquine, chroloquine, azithromycin, dexamethasone, niacin, steroids (e.g., corticosteroids, etc.), vitamins (e.g., vitamin C, vitamin D, vitamin B, etc.), stem cells, baloxavir, chloroquine phosphate, lopinavir, ritonavir, neuraminidase inhibitors (e.g., oseltamivir), remedesivir, ascorbic acid, methylprednisolone, nitric oxide, sarilumab, sirolimus, tocilizumab, angiotensin converting enzyme (ACE) inhibitors, angiotensin receptor blockers (ARBs) (e.g., angiotensin II receptor blockers), ibuprofen, indomethacin, and niclosamide.

Non-limiting examples of an ACE inhibitor can include, but are not limited to, Alacepril, Captopril, Zefnopril, Enalapril, Ramipril, Quinapril, Perindopril, LIsinopril, Benazepril, Imidapril, Tradolapril, Cilazapril, Fosinopril, anti-hypertensive peptide(s), Arfalasin, Casokinin, Lactokinin, Lactotripeptides (e.g., Val-Pro-Pro or Ile-Pro-Pro), etc. Non-limiting examples of an angiotensin receptor inhibitor/blocker, such as Angiotensin receptor II blocker, can include, but are not limited to, Azilsartan, Candesartan, Eprosartan, Irbesartan, Losartan, Olmesartan, Telmisartan, Valsartan, etc.

A therapeutic composition may comprise a cannabinoid composition and one or more therapeutic compounds (e.g., at least 1, 2, 3, 4, 5, 6, 7, 8, 9, 10, or more different types of therapeutic compounds), such as the compounds described above.

In a therapeutic composition, a cannabinoid composition (or compound as used interchangeably herein) as disclosed herein (e.g., one or more cannabinoid compounds, such as cannabidiol) and/or an additional therapeutic compound (e.g., one or more additional non-cannabinoid compounds) may be encapsulated. In some cases, the cannabinoid composition may be encapsulated, and the additional therapeutic compound may not and need not be encapsulated. In some cases, the cannabinoid composition may not and need not be encapsulated, and the additional therapeutic compound may be encapsulated. In some cases, the cannabinoid composition and the additional therapeutic compound may be encapsulated. For example, the cannabinoid composition and the additional therapeutic compound may be encapsulated in the same microcapsule. In another example, the cannabinoid composition and the additional therapeutic compound may be encapsulated in different microcapsules. In some cases, the therapeutic compound may comprise a plurality of additional non-cannabinoid therapeutic compounds, and at least a first non-cannabinoid therapeutic compound may be encapsulated while at least a second non-cannabinoid therapeutic compound may not and need not be encapsulated.

A therapeutic composition may comprise a plurality of therapeutic doses. The plurality of therapeutic doses may be administered to a subject in need thereof via the same route (e.g., via oral administration). Alternatively or in addition to, the plurality of therapeutic doses may be administered to the subject in need thereof via different routes (e.g., a first therapeutic dose via oral administration and a second therapeutic dose via nebulization). The plurality of therapeutic doses may be administered to the subject at the same time. Alternatively or in addition to, the plurality of therapeutic doses may be administered to the subject at different times. In some cases, the first therapeutic dose may comprise the cannabinoid composition(s)/compound(s) as disclosed herein, while the second therapeutic dose may comprise the additional therapeutic composition(s)/compound(s) as disclosed herein. The first therapeutic dose may be administered to the subject prior to, concurrently with (or substantially at the same time), or subsequent to administration of the second therapeutic dose to the subject.

In some cases, the therapeutic composition as disclosed herein can comprise a first plurality of microcapsules (FM) comprising the cannabinoid composition and a second plurality of microcapsules (SM) comprising the additional therapeutic compound in a weight ratio (FM:SM) of at least about 100:1, at least about 100:2, at least about 100:3, at least about 100:4, at least about 100:5, at least about 100:6, at least about 100:7, at least about 100:8, at least about 100:9, at least about 100:10, at least about 100:20, at least about 100:30, at least about 100:40, at least about 100:50, at least about 100:60, at least about 100:70, at least about 100:80, at least about 100:90, at least about 100:100, at least about 90:100, at least about 80:100, at least about 70:100, at least about 60:100, at least about 50:100, at least about 40:100, at least about 30:100, at least about 20:100, at least about 10:100, at least about 9:100, at least about 8:100, at least about 7:100, at least about 6:100, at least about 5:100, at least about 4:100, at least about 3:100, at least about 2:100, or at least about 1. The weight ratio (FM:SM) can be at most about 100:1, at most about 100:2, at most about 100:3, at most about 100:4, at most about 100:5, at most about 100:6, at most about 100:7, at most about 100:8, at most about 100:9, at most about 100:10, at most about 100:20, at most about 100:30, at most about 100:40, at most about 100:50, at most about 100:60, at most about 100:70, at most about 100:80, at most about 100:90, at most about 100:100, at most about 90:100, at most about 80:100, at most about 70:100, at most about 60:100, at most about 50:100, at most about 40:100, at most about 30:100, at most about 20:100, at most about 10:100, at most about 9:100, at most about 8:100, at most about 7:100, at most about 6:100, at most about 5:100, at most about 4:100, at most about 3:100, at most about 2:100, or at most about 1.

In some cases, the therapeutic composition as disclosed herein can comprise the first plurality of microcapsules (FM) comprising the cannabinoid composition and the second plurality of microcapsules (SM) comprising the additional therapeutic compound in a volume ratio (FM:SM) of at least about 100:1, at least about 100:2, at least about 100:3, at least about 100:4, at least about 100:5, at least about 100:6, at least about 100:7, at least about 100:8, at least about 100:9, at least about 100:10, at least about 100:20, at least about 100:30, at least about 100:40, at least about 100:50, at least about 100:60, at least about 100:70, at least about 100:80, at least about 100:90, at least about 100:100, at least about 90:100, at least about 80:100, at least about 70:100, at least about 60:100, at least about 50:100, at least about 40:100, at least about 30:100, at least about 20:100, at least about 10:100, at least about 9:100, at least about 8:100, at least about 7:100, at least about 6:100, at least about 5:100, at least about 4:100, at least about 3:100, at least about 2:100, or at least about 1. The volume ratio (FM:SM) can be at most about 100:1, at most about 100:2, at most about 100:3, at most about 100:4, at most about 100:5, at most about 100:6, at most about 100:7, at most about 100:8, at most about 100:9, at most about 100:10, at most about 100:20, at most about 100:30, at most about 100:40, at most about 100:50, at most about 100:60, at most about 100:70, at most about 100:80, at most about 100:90, at most about 100:100, at most about 90:100, at most about 80:100, at most about 70:100, at most about 60:100, at most about 50:100, at most about 40:100, at most about 30:100, at most about 20:100, at most about 10:100, at most about 9:100, at most about 8:100, at most about 7:100, at most about 6:100, at most about 5:100, at most about 4:100, at most about 3:100, at most about 2:100, or at most about 1.

The therapeutic composition as disclosed herein can comprise the cannabinoid composition (CC) and the additional therapeutic compound (ATC) in a weight ratio (CC:ATC) of at least about 100:1, at least about 100:2, at least about 100:3, at least about 100:4, at least about 100:5, at least about 100:6, at least about 100:7, at least about 100:8, at least about 100:9, at least about 100:10, at least about 100:20, at least about 100:30, at least about 100:40, at least about 100:50, at least about 100:60, at least about 100:70, at least about 100:80, at least about 100:90, at least about 100:100, at least about 90:100, at least about 80:100, at least about 70:100, at least about 60:100, at least about 50:100, at least about 40:100, at least about 30:100, at least about 20:100, at least about 10:100, at least about 9:100, at least about 8:100, at least about 7:100, at least about 6:100, at least about 5:100, at least about 4:100, at least about 3:100, at least about 2:100, or at least about 1. The weight ratio (CC:ATC) can be at most about 100:1, at most about 100:2, at most about 100:3, at most about 100:4, at most about 100:5, at most about 100:6, at most about 100:7, at most about 100:8, at most about 100:9, at most about 100:10, at most about 100:20, at most about 100:30, at most about 100:40, at most about 100:50, at most about 100:60, at most about 100:70, at most about 100:80, at most about 100:90, at most about 100:100, at most about 90:100, at most about 80:100, at most about 70:100, at most about 60:100, at most about 50:100, at most about 40:100, at most about 30:100, at most about 20:100, at most about 10:100, at most about 9:100, at most about 8:100, at most about 7:100, at most about 6:100, at most about 5:100, at most about 4:100, at most about 3:100, at most about 2:100, or at most about 1.

In some cases, the non-cannabinoid therapeutic composition/compound as disclosed herein may not be terpenes and/or essential oils. In some cases, the additional therapeutic composition/compound as disclosed herein may not be terpenes and/or essential oils.

A therapeutic composition may comprise terpenes, essential oils, and other desirable compounds.

In an example, a therapeutic composition comprises a cannabinoid compound in encapsulated form. In an example, a therapeutic composition comprises a cannabinoid compound in non-encapsulated form. In an example, a therapeutic composition comprises a cannabinoid compound and hydroxychloroquine in encapsulated form. In an example, a therapeutic composition comprises a cannabinoid compound and hydroxychloroquine in non-encapsulated form. In an example, a therapeutic composition comprises a cannabinoid compound and chloroquine in encapsulated form. In an example, a therapeutic composition comprises a cannabinoid compound and chloroquine in non-encapsulated form. In an example, a therapeutic composition comprises a cannabinoid compound, hydroxychloroquine and/or chloroquine, and azithromycin in encapsulated form. In an example, a therapeutic composition comprises a cannabinoid compound, hydroxychloroquine and/or chloroquine, and azithromycin in non-encapsulated form. In an example, a therapeutic composition comprises a cannabinoid compound and vitamin C in encapsulated form. In an example, a therapeutic composition comprises a cannabinoid compound and vitamin C in non-encapsulated form. In an example, a therapeutic composition comprises a cannabinoid compound and vitamins C, D, and B in encapsulated form. In an example, a therapeutic composition comprises a cannabinoid compound and vitamins C, D, and B in non-encapsulated form. In an example, a therapeutic composition comprises a cannabinoid compound and niacin in encapsulated form. In an example, a therapeutic composition comprises a cannabinoid compound and niacin in non-encapsulated form. In an example, a therapeutic composition comprises a cannabinoid compound and a steroid in encapsulated form. In an example, a therapeutic composition comprises a cannabinoid compound and a steroid in non-encapsulated form. In an example, a therapeutic composition comprises a cannabinoid compound and dexamethasone in encapsulated form. In an example, a therapeutic composition comprises a cannabinoid compound and dexamethasone in non-encapsulated form.

A therapeutic composition may be administered prior to, substantially simultaneously with or simultaneously with, or subsequent to another treatment (e.g., chemotherapy).

Beneficially, such therapeutic compositions may treat infections by cellular protection, anti-viral, anti-bacterial, anti-inflammatory modulation, immune modulation, expectorative inducement, and/or systemic homeostatic enhancement. The presence of the cannabinoid compound in the therapeutic composition may counteract or treat symptoms caused or exacerbated by an additional therapeutic compound. For example, hydroxychloroquine or chloroquine may present risk factors to subjects with heart issues, and the cannabinoid compound may facilitate protection of the heart and overall homeostasis of the subject's body. The cannabinoid compound (e.g., cannabidiol) can attenuate cardiac dysfunction, oxidative stress, fibrosis, and inflammatory and cell death signaling pathways in diabetic cardiomyopathy. The cannabinoid compound may help with arrythmias. For example, where a therapeutic composition comprises a cannabinoid compound and a chemotherapy agent, such as doxorubicin, the cannabinoid compound may decrease damage to the body from the chemotherapy agent and enhance efficacy of the chemotherapy. Cannabinoid compounds, such as doxorubicin, can limit cardiotoxicity when co-administered in doxorubicin (DOX) chemotherapy treatment.

The presence of the cannabinoid compound in the therapeutic composition may counteract or treat symptoms caused or exacerbated by a virus. For example, subject may suffer from acute respiratory disease syndrome (ARDS) caused by one or more viruses (e.g., coronavirus). For example, diseases caused by a virus described herein (e.g., COVID-19) can lead to Cytokine Storm Syndrome (CSS). The cannabinoid compound can prevent CSS, attenuate symptoms caused by CSS, and improve overall lung function. In a 15-person 60-day study, subjects were provided with microencapsulated cannabinoid compositions. A blood analysis revealed 12-15% reduction in three primary inflammatory markers also associated with CSS.

The present invention provides a therapeutic composition comprising a plurality of microcapsules, wherein one or more therapeutic compositions are present in an amount of at least about one microgram. A therapeutic composition may be in non-encapsulated form, present in an amount of at least about one microgram. In other embodiments, the present invention provides food products that are rich in therapeutic compositions.

### Microfluidic Encapsulation and Delivery

The compositions of the present disclosure can comprise microcapsules. Microcapsules can comprise components discussed elsewhere in this disclosure, such as the therapeutic compositions described herein. Microcapsules can comprise a mushroom, fulvic acid, L-Theanine, Fish Oil, pregnenolone, phenyl ethyl amine (PEA), tulsi, lemon balm, passion flower, terpene compounds, blue lotus, cacao, maca, schizandra, Siberian ginseng, kava, skullcap, valerian, hops, California poppy, catuba, epidmedium, pao d'arco, ashwaganda, ginko, albiza, reishi, lion's mane, maitake, chaga, vitamin C, turmeric, cannabinoid compounds, cannabidiol (CBD), tetrahydrocannabinol (THC), bioperine, and xanthohumol oil-based compounds, and others, in microencapsulated form. In some cases, compositions can be encapsulated without the use of liposomes. In some cases, compositions can be encapsulated without the use of micelles. In some cases, compositions can be encapsulated without the use of liposomes or micelles. Compounds of the composition can exist within a microcapsule in forms including but not limited to liquid, gel, semi-solid, and solid. Microcapsules of compositions disclosed herein can further be processed into forms including but not limited to solids, powders, liquids, suspensions, gels, tablets, foods, lotions, cosmetics, and other forms discussed in this disclosure.

Microencapsulation can be performed with a microencapsulation device, including microfluidic droplet generation or encapsulation devices. An exemplary microencapsulation device is described, for example, in U.S. Pat. No. 7,482,152, incorporated here by reference in its entirety. Microfluidic droplets or emulsions (e.g., microcapsules) can be generated by flow of a fluid to be encapsulated with an immiscible carrier fluid. For example, an oil fluid to be encapsulated can be flowed with an aqueous carrier fluid, or an aqueous fluid to be encapsulated can be flowed with an oil carrier fluid. Air can also be used as a fluid. Microfluidic droplet generators useful for microencapsulation include those employing co-flowing streams, cross-flowing streams (e.g., flow of streams at a T-junction), flow focusing, flow through perforated plates, and flow through nozzles. Droplet size can be controlled by parameters including device geometry, relative flow rates of the fluid streams, and operating pressure.

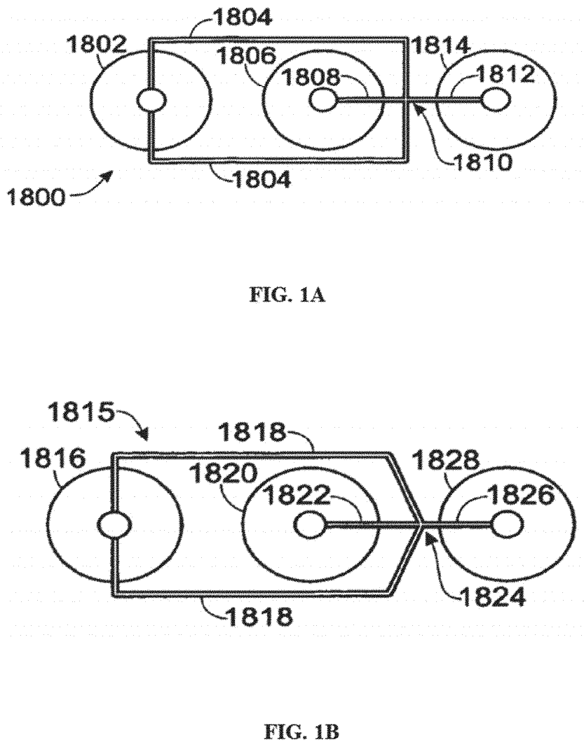

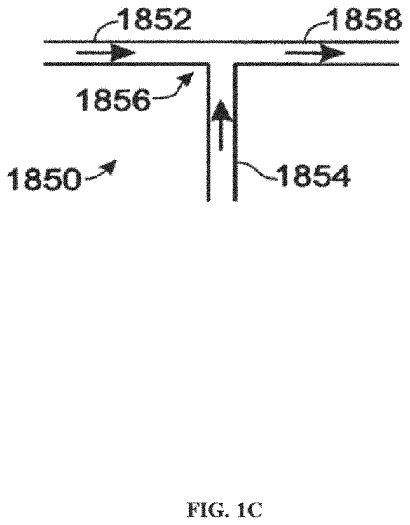

FIG. 1A shows an example of a droplet generator. In configuration 1800, a first phase (e.g., oil) from a first fluid chamber 1802 is transferred through two branches of a fluid channel section 1804. The first phase from the first fluid chamber 1802 intersects with a second phase (e.g., aqueous phase) from a second fluid chamber 1806, which is transferred along a fluid channel section 1808 to an intersection 1810 with the fluid channel section 1804. For example, the first phase from the first fluid chamber 1802 arrives at intersection 1810 from two different and substantially opposite directions, whereas the second phase arrives at the intersection along only a single path that is substantially perpendicular to both directions of travel of the arriving first phase fluid. At intersection 1810, droplets in the second phase are generated in a first phase background (e.g., a water-in-oil emulsion) and transferred along a fluid channel section 1812 to a third fluid chamber 1814, where the emulsion can be temporarily stored and/or transferred to another location. The phases can be reversed, for example, such that the droplets in the first phase are generated in a second phase background (e.g., an oil-in-water emulsion).

FIG. 1B shows another example of a droplet generator. In configuration 1815, a first phase (e.g., oil) from a first fluid chamber 1816 is transferred through two branches of a fluid channel section 1818. Fluid channel section 1818 intersects with a fluid channel section 1822 that transfers a second phase (e.g., aqueous) fluid from a second fluid chamber 1820, at intersection 1824. As in configuration 1800 of FIG. 1A, the first phase from the first fluid chamber 1816 arrives at intersection 1810 from two different directions, but unlike in configuration 1800, the fluid does not arrive from substantially opposite (antiparallel) directions. Rather, the branches of channel section 1818 each intersect channel section 1822 at a non-perpendicular angle, which is depicted as approximately 60 degrees in FIG. 1B. In general, configuration 1815 may include first phase fluid channels that intersect a second phase fluid channel at any desired angle or angles. The first phase fluid flowing through channel sections 1818 and the second phase fluid flowing through channel section 1822 can combine to form an emulsion of droplets in the second phase suspended in a first phase background (e.g., water-in-oil emulsion). As in the case of configuration 1800, the droplets then may be transferred along a fluid channel section 1826 to a third fluid chamber 1828, for storage and/or transfer to another location. The phases can be reversed, for example, such that the droplets in the first phase are generated in a second phase background (e.g., an oil-in-water emulsion).

FIG. 1C shows an example of a microfluidic structure. The arrows within the depicted fluid channels indicate the direction of fluid flow within each channel. Although fluid chambers for receiving and/or storing oil, water, and any generated emulsion are not depicted, these chambers or at least some source of oil and aqueous fluid would be present in a cartridge containing any of the depicted configurations. The fluid channels and any associated chambers may be formed by any suitable method, such as injection molding complementary sections of thermoplastic as described previously. In a T-junction configuration 1850, a first phase fluid traveling along channel 1852 intersects with a second phase fluid traveling along channel 1854 at intersection 1856, to produce second phase-in-first phase emulsions (e.g., water-in-oil, of-in-water, etc.) that travels along outgoing fluid channel 1858. Droplets formed in the T-junction configuration 1850 may be formed primarily by a shear mechanism rather than primarily by a compression mechanism. However, droplet formation may depend on many factors, including the channel diameters, fluid velocities, and fluid viscosities.

Microencapsulation can be performed at a range of operating parameters, such as different flow rates or pressures. Microencapsulation can be conducted at a pressure of at least about 10 pounds per square inch (psi), 20 psi, 30 psi, 40 psi, 50 psi, 60 psi, 70 psi, 80 psi, 90 psi, 100 psi, 200 psi, 300 psi, 400 psi, 500 psi, 600 psi, 700 psi, 800 psi, 900 psi, 1000 psi, 2000 psi, 3000 psi, 4000 psi, 5000 psi, 6000 psi, 7000 psi, 8000 psi, 9000 psi, 10000 psi, 15000 psi, 20000 psi, 25000 psi, 30000 psi, 35000 psi, 40000 psi, 45000 psi, 50000 psi, or more. Microencapsulation can be conducted at a pressure of at most about 10 pounds per square inch (psi), 20 psi, 30 psi, 40 psi, 50 psi, 60 psi, 70 psi, 80 psi, 90 psi, 100 psi, 200 psi, 300 psi, 400 psi, 500 psi, 600 psi, 700 psi, 800 psi, 900 psi, 1000 psi, 2000 psi, 3000 psi, 4000 psi, 5000 psi, 6000 psi, 7000 psi, 8000 psi, 9000 psi, 10000 psi, 15000 psi, 20000 psi, 25000 psi, 30000 psi, 35000 psi, 40000 psi, 45000 psi, or 50000 psi. Microencapsulation can be conducted at a pressure of about 10 pounds per square inch (psi), 20 psi, 30 psi, 40 psi, 50 psi, 60 psi, 70 psi, 80 psi, 90 psi, 100 psi, 200 psi, 300 psi, 400 psi, 500 psi, 600 psi, 700 psi, 800 psi, 900 psi, 1000 psi, 2000 psi, 3000 psi, 4000 psi, 5000 psi, 6000 psi, 7000 psi, 8000 psi, 9000 psi, 10000 psi, 15000 psi, 20000 psi, 25000 psi, 30000 psi, 35000 psi, 40000 psi, 45000 psi, 50000 psi, or more. Microencapsulation can be conducted at a flow rate of at least about 1 milliliter per minute (mL/min), 2 mL/min 3 mL/min, 4 mL/min, 5 mL/min, 6 mL/min, 7 mL/min, 8 mL/min, 9 mL/min, 10 mL/min, 20 mL/min, 30 mL/min, 40 mL/min, 50 mL/min, 60 mL/min, 70 mL/min, 80 mL/min, 90 mL/min, 100 mL/min, 110 mL/min, 120 mL/min, 130 mL/min, 140 mL/min, 150 mL/min, 160 mL/min, 170 mL/min, 180 mL/min, 190 mL/min, 200 mL/min, 210 mL/min, 220 mL/min, 230 mL/min, 240 mL/min, 250 mL/min, 260 mL/min, 270 mL/min, 280 mL/min, 290 mL/min, 300 mL/min, 310 mL/min, 320 mL/min, 330 mL/min, 340 mL/min, 350 mL/min, 360 mL/min, 370 mL/min, 380 mL/min, 390 mL/min, 400 mL/min, 410 mL/min, 420 mL/min, 430 mL/min, 440 mL/min, 450 mL/min, 460 mL/min, 470 mL/min, 480 mL/min, 490 mL/min, 500 mL/min, or more. Microencapsulation can be conducted at a flow rate of at most about 1 milliliter per minute (mL/min), 2 mL/min 3 mL/min, 4 mL/min, 5 mL/min, 6 mL/min, 7 mL/min, 8 mL/min, 9 mL/min, 10 mL/min, 20 mL/min, 30 mL/min, 40 mL/min, 50 mL/min, 60 mL/min, 70 mL/min, 80 mL/min, 90 mL/min, 100 mL/min, 110 mL/min, 120 mL/min, 130 mL/min, 140 mL/min, 150 mL/min, 160 mL/min, 170 mL/min, 180 mL/min, 190 mL/min, 200 mL/min, 210 mL/min, 220 mL/min, 230 mL/min, 240 mL/min, 250 mL/min, 260 mL/min, 270 mL/min, 280 mL/min, 290 mL/min, 300 mL/min, 310 mL/min, 320 mL/min, 330 mL/min, 340 mL/min, 350 mL/min, 360 mL/min, 370 mL/min, 380 mL/min, 390 mL/min, 400 mL/min, 410 mL/min, 420 mL/min, 430 mL/min, 440 mL/min, 450 mL/min, 460 mL/min, 470 mL/min, 480 mL/min, 490 mL/min, or 500 mL/min. Microencapsulation can be conducted at a flow rate of about 1 milliliter per minute (mL/min), 2 mL/min 3 mL/min, 4 mL/min, 5 mL/min, 6 mL/min, 7 mL/min, 8 mL/min, 9 mL/min, 10 mL/min, 20 mL/min, 30 mL/min, 40 mL/min, 50 mL/min, 60 mL/min, 70 mL/min, 80 mL/min, 90 mL/min, 100 mL/min, 110 mL/min, 120 mL/min, 130 mL/min, 140 mL/min, 150 mL/min, 160 mL/min, 170 mL/min, 180 mL/min, 190 mL/min, 200 mL/min, 210 mL/min, 220 mL/min, 230 mL/min, 240 mL/min, 250 mL/min, 260 mL/min, 270 mL/min, 280 mL/min, 290 mL/min, 300 mL/min, 310 mL/min, 320 mL/min, 330 mL/min, 340 mL/min, 350 mL/min, 360 mL/min, 370 mL/min, 380 mL/min, 390 mL/min, 400 mL/min, 410 mL/min, 420 mL/min, 430 mL/min, 440 mL/min, 450 mL/min, 460 mL/min, 470 mL/min, 480 mL/min, 490 mL/min, 500 mL/min, or more.

Droplet generators can employ multiple parallel droplet generation operations in parallel. For example, a droplet generator (e.g., a plate, a device with channels) can employ at least 1, 2, 3, 4, 5, 6, 7, 8, 9, 10, 15, 20, 25, 30, 35, 40, 45, 50, 55, 60, 65, 70, 75, 80, 85, 90, 95, 100, or more droplet generating features (e.g., holes, channels, nozzles). A droplet generator can employ at most 1, 2, 3, 4, 5, 6, 7, 8, 9, 10, 15, 20, 25, 30, 35, 40, 45, 50, 55, 60, 65, 70, 75, 80, 85, 90, 95, or 100 droplet generating features. A droplet generator can employ about 1, 2, 3, 4, 5, 6, 7, 8, 9, 10, 15, 20, 25, 30, 35, 40, 45, 50, 55, 60, 65, 70, 75, 80, 85, 90, 95, 100, or more droplet generating features.

Microencapsulation can be performed via an emulsification process. For example, compositions can be emulsified in a mixer, such as an agitator, impeller, centrifugal mixer, or high-shear mixer. High-shear mixers can include batch high-shear mixers and inline high-shear mixers (e.g., rotor-stator mixers). Emulsification can also be conducted without a mixer, by combining fluids thermodynamically favored to form an emulsion, optionally with the aid of one or more emulsifiers or surfactants.

Microencapsulation processes can be conducted with the aid of one or more emulsifiers or surfactants. Emulsifiers and surfactants can include but are not limited to saponins (e.g., quillaja tree extract such as Q-NATURALE®, yucca extract), lecithin, soy lecithin, mustard seed hull extract, sodium stearoyl lactylate, polysorbate 20, and combinations thereof.

Microcapsules can comprise one or more stabilizers or gelling agents, which can be used to stabilize a microcapsule or emulsion. Stabilizers or gelling agents can include but are not limited to alginate (also algin or alginic acid) and agar. Alginate can be used in a variety of forms, including but not limited to inorganic salts such as sodium alginate, potassium alginate, calcium alginate, and combinations thereof. Alginate can be derived from sources such as seaweed (e.g., Macrocystis pyrifera, Ascophyllum nodosum, Laminaria spp.) or bacteria (e.g., Pseudomonas spp., Azotobacter spp.). Cross-linking agents or solutions, such as calcium chloride, can be used to stabilize or gel microcapsules.

Microcapsules can be characterized by a size (e.g., a diameter). The microcapsule size can be about 0.154 micrometers. The microcapsule size can be less than or equal to about 0.154 micrometers. The microcapsule size can be greater than or equal to about 0.154 micrometers. The microcapsule size can be about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 150, 200, 250, 300, 350, 400, 450, or 500 micrometers. The microcapsule size can be less than or equal to about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 150, 200, 250, 300, 350, 400, 450, or 500 micrometers. The microcapsule size can be greater than or equal to about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 150, 200, 250, 300, 350, 400, 450, or 500 micrometers. The microcapsule size can be from about 0.1 to about 0.2 micrometers. The microcapsule size can be from about 0.05 to about 0.25 micrometers. The microcapsule size can be from about 0.05 to about 0.55 micrometers. The microcapsule size can be from about 0.05 to about 1 micrometers. The size distribution in a population of microcapsules can be homogeneous or substantially homogeneous. For example, a population of microcapsules can be characterized by dispersity, or polydispersity index (PDI), of less than or equal to about 20, 19, 18, 17, 16, 16, 15, 14, 13, 12, 11, 10, 9, 8, 7, 6, 5, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.45, 1.40, 1.35, 1.30, 1.25, 1.20, 1.15, 1.14, 1.13, 1.12, 1.11, 1.10, 1.09, 1.08, 1.07, 1.06, 1.05, 1.04, 1.03, 1.02, 1.01, or 1.00.

### Compositions

Therapeutic compositions of the present disclosure may comprise a variety of compounds, such as hydroxychloroquine, chroloquine, azithromycin, dexamethasone, niacin, steroids (e.g., corticosteroids, etc.), vitamins (e.g., vitamin C, vitamin D, vitamin B, etc.), stem cells, baloxavir, chloroquine phosphate, lopinavir, ritonavir, neuraminidase inhibitors (e.g., oseltamivir), remedesivir, ascorbic acid, methylprednisolone, nitric oxide, sarilumab, sirolimus, tocilizumab, angiotensin converting enzyme (ACE) inhibitors, angiotensin II receptor blockers (ARBs), ibuprofen, indomethacin, and niclosamide, mushroom, fulvic acid, L-Theanine, Fish Oil, pregnenolone, phenyl ethyl amine (PEA), tulsi, lemon balm, passion flower, terpene compounds, blue lotus, cacao, maca, schizandra, Siberian ginseng, kava, skullcap, valerian, hops, California poppy, catuba, epidmedium, pao d'arco, ashwaganda, ginko, albiza, reishi, lion's mane, maitake, chaga, vitamin C, turmeric, cannabinoid compounds, cannabidiol (CBD), tetrahydrocannabinol (THC), bioperine, xanthohumol oil-based compounds, and others, and/or a combination thereof.

Cannabinoids utilized in the compositions disclosed herein include but are not limited to cannabigerol-type (CBG), cannabigerolic acid (CBGA), cannabigerolic acid monomethylether (CBGAM), cannabigerol monomethyl ether (CBGM), cannabichromene-type (CBC), cannabichromanon (CBCN), cannabichromenic acid (CBCA), cannabichromevarin-type (CBCV), cannabichromevarinic acid (CBCVA), cannabidiol-type (CBD), tetrahydrocannabinol-type (THC), iso-tetrahydrocannabinol-type (iso-THC), cannabinol-type (CBN), cannabinolic acid (CBNA), cannabinol methylether (CBNM), cannabinol-C₄ (CBN-C₄), cannabinol-C₂ (CBN-C₂), cannabiorcol (CBN-C₁), cannabinodiol (CBND), cannabielsoin-type (CBE), cannabielsoic acid A (CBEA-A), cannabielsoic acid B (CBEA-B), cannabicyclol-type (CBL), cannabicyclolic acid (CBLA), cannabicyclovarin (CBLV), cannabicitran-type (CBT), cannabitriol, cannabitriolvarin (CBTV), ethoxy-cannabitiolvarin (CBTVE), cannabivarin-type (CBV), cannabinodivarin (CBVD), tetrahydrocannabivarin-type (THCV), cannabidivarin-type (CBDV), cannabigerovarin-type (CBGV), cannabigerovarinic acid (CBGVA), cannabifuran (CBF), dehydrocannabifuran (DCBF), and cannabiripsol (CBR) cannabinoids.

Cannabinoids used in compositions of the present disclosure can be derived from various sources, including but not limited to hemp (e.g. hemp stalk, hemp stem, hemp seed), cannabis (e.g., cannabis flower, cannabis leaf, cannabis stalk, cannabis stem, cannabis seed), Echinacea purpurea, Echinacea angustifolia, Echinacea pallida, Acmella oleracea, Helichrysum umbraculigerum, Radula marginata, kava, black truffle, Syzygium aromaticum (cloves), Rosmarinus oficinalis, basil, oregano, black pepper, lavender, true cinnamon, malabathrum, cananga odorata, copaifera spp., and hops.

Encapsulated cannabinoids can be present in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated cannabinoids can be present in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated cannabinoids can be present in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated cannabinoids can be present in a quantity of from about 1 to about 10 micrograms per microcapsule. Encapsulated cannabinoids can be present in a quantity of at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule. Encapsulated cannabinoids can be present in a quantity of at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule. Encapsulated cannabinoids can be present in a quantity of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule.

Cannabinoids can be present in any product, such as a food product or any therapeutic composition, in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabinoids can be present in any product, such as a food product or any therapeutic composition, in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabinoids can be present in any product, such as a food product or any therapeutic composition, in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabinoids can be present in any product, such as a food product or any therapeutic composition, in a quantity of from about 50 to about 150 milligrams. Cannabinoids can be present in any product, such as a food product or any therapeutic composition, in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Cannabinoids can be present in any product, such as a food product or any therapeutic composition, in a quantity of at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Cannabinoids can be present in any product, such as a food product or any therapeutic composition, in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

The cannabinoids of the compositions disclosed herein can comprise cannabidiol-class compounds, including but not limited to cannabidiol (CBD), cannabidiolic acid (CBDA), cannabidiol monomethylether (CBDM), cannabidiol-C₄ (CBD-C₄), cannabidivarin (CBDV), cannabidivarinic acid (CBDVA), cannabidiorcol (CBD-C₁), and combinations thereof. CBD can comprise delta-1-cannabidiol, delta-2- cannabidiol, delta-3- cannabidiol, delta-3,7- cannabidiol, delta-4- cannabidiol, delta-5- cannabidiol, delta-6- cannabidiol, and combinations thereof.

Encapsulated cannabidiol compounds can be present in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated cannabidiol compounds can be present in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated cannabidiol compounds can be present in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated cannabidiol compounds can be present in a quantity of from about 1 to about 10 micrograms per microcapsule. Encapsulated cannabidiol compounds can be present in a quantity of at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule. Encapsulated cannabidiol compounds can be present in a quantity of at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule. Encapsulated cannabidiol compounds can be present in a quantity of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule.

Cannabidiol compounds can be present in any product, such as a food product or any therapeutic composition, in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabidiol compounds can be present in any product, such as a food product or any therapeutic composition, in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabidiol compounds can be present in any product, such as a food product or any therapeutic composition, in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabidiol compounds can be present in any product, such as a food product or any therapeutic composition, in a quantity of from about 50 to about 150 milligrams. Cannabidiol compounds can be present in any product, such as a food product or any therapeutic composition, in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Cannabidiol compounds can be present in any product, such as a food product or any therapeutic composition, in a quantity of at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Cannabidiol compounds can be present in any product, such as a food product or any therapeutic composition, in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

The compositions of the present disclosure can comprise tetrahydrocannabinol (THC) as a type of cannabinoids. THC can comprise delta-9-THC, delta-8-THC, and combinations thereof. THC can comprise delta-6a,7-tetrahydrocannabinol, delta-7-tetrahydrocannabinol, delta-8-tetrahydrocannabinol, delta-9,11-tetrahydrocannabinol, delta-9-tetrahydrocannabinol, delta-10-tetrahydrocannabinol, delta-6a,10a-tetrahydrocannabinol, and combinations thereof. Delta-9-tetrahydrocannabinol can comprise stereoisomers including (6aR,10aR)-delta-9-tetrahydrocannabinol, (6aS,10aR)-delta-9-tetrahydrocannabinol, (6aS,10aS)-delta-9-tetrahydrocannabinol, (6aR,10aS)-delta-9-tetrahydrocannabinol, and combinations thereof.

In cases where the compositions comprise microcapsules, THC compounds can be present in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated THC compounds can be present in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated THC compounds can be present in a quantity of from about 1 to about 10 micrograms per microcapsule. Encapsulated THC compounds can be present in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated THC compounds can be present in a quantity of at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule. Encapsulated THC compounds can be present in a quantity of at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule. Encapsulated THC compounds can be present in a quantity of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule.

THC compounds can be present in any product, such as a food product or any therapeutic composition, in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). THC compounds can be present in any product, such as a food product or any therapeutic composition, in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). THC compounds can be present in any product, such as a food product or any therapeutic composition, in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). THC compounds can be present in any product, such as a food product or any therapeutic composition, in a quantity of from about 50 to about 150 milligrams. THC compounds can be present in any product, such as a food product or any therapeutic composition, in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. THC compounds can be present in any product, such as a food product or any therapeutic composition, in a quantity of at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. THC compounds can be present in any product, such as a food product or any therapeutic composition, in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

In some cases, a composition of the present disclosure does not contain a psychoactive amount of THC. For example, cannabinoids in compositions of the present disclosure can contain less than 100%, 90%, 80%, 70%, 60%, 50%, 40%, 30%, 20%, 10%, 5%, 1%, 0.7%, 0.5%, 0.3%, or 0.1% THC relative to the total quantity of cannabinoid compounds. In some cases, the ratio of a non-THC cannabinoid (e.g., cannabidiol) to THC in a composition of the present disclosure is greater than or equal to about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, or 100:1. In some cases, compositions of the present disclosure contain less than 0.3% THC.

The compositions of the present disclosure can comprise one or more additional therapeutic compositions as disclosed herein (e.g., one or more non-cannabinoid compounds, such as hydroxychloroquine, chroloquine, azithromycin, dexamethasone, steroids, vitamins C, vitamin D, stem cells, etc.). The additional therapeutic composition(s) can be present in a quantity of at least about 0.01, 0.02, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 500, or 1,000 micrograms per a therapeutic composition. The additional therapeutic composition(s) can be present in a quantity of at most about 0.01, 0.02, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 500, or 1,000 micrograms per a therapeutic composition. The additional therapeutic composition(s) can be present in a quantity of at about 0.01, 0.02, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 500, or 1,000 micrograms per a therapeutic composition. The additional therapeutic composition(s) can be present in a quantity of at least about 00.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a therapeutic composition. The additional therapeutic composition(s) can be present in a quantity of at most about 00.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a therapeutic composition. The additional therapeutic composition(s) can be present in a quantity of about 00.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a therapeutic composition.

Encapsulated additional therapeutic composition(s) can be present in a quantity of at least about 0.01, 0.02, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 500, or 1,000 micrograms per microcapsule. Encapsulated additional therapeutic composition(s) can be present in a quantity of at most about 0.01, 0.02, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 500, or 1,000 micrograms per microcapsule. Encapsulated additional therapeutic composition(s) can be present in a quantity of at about 0.01, 0.02, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 500, or 1,000 micrograms per microcapsule. Encapsulated additional therapeutic composition(s) can be present in a quantity of at least about 00.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule. Encapsulated additional therapeutic composition(s) can be present in a quantity of at most about 00.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule. Encapsulated additional therapeutic composition(s) can be present in a quantity of about 00.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule.

The compositions of the present disclosure can comprise one or more terpene compounds, including but not limited to terpenoids such as monoterpenoids, sesquiterpenoids, diterpenoids, and triterpenoids. Terpenes can be acyclic, monocyclic, or polycyclic. Terpenes can include but are not limited to myrcene, limonene, linalool, trans-ocimene, cis-ocimene, alpha-pinene, beta-pinene, alpha-humulene (alpha-caryophyllene), beta-caryophyllene, delta-3-carene, trans-gamma-bisabolene, cis-gamma-bisabolene, trans-alpha-farnesene, cis-beta-farnesene, beta-fenchol, beta-phellandrene, guajol, alpha-gualene, alpha-eudesmol, beta-eudesmol, gamma-eudesmol, terpinolene, alpha-selinene, beta-selinene, alpha-terpineol, fenchone, camphene, cis-sabinene hydrate, alpha-trans-bergamotene, alpha-cis-bergamotene, borneol, gamma-curcumene, alpha-thujene, epi-alpha-bisabolol, ipsdienol, alpha-ylangene, beta-elemene, gamma-muurolene, alpha-cadinene, alpha-longipinene, caryophyllene oxide, and combinations thereof.

Encapsulated terpenes can be present in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated terpenes can be present in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated terpenes can be present in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated terpene compounds can be present in a quantity of from about 1 to about 10 micrograms per microcapsule. Encapsulated terpenes can be present in a quantity of at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule. Encapsulated terpenes can be present in a quantity of at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule. Encapsulated terpenes can be present in a quantity of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule.

Terpene compounds can be present in any product, such as a food product or any therapeutic composition, in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Terpene compounds can be present in any product, such as a food product or any therapeutic composition, in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Terpene compounds can be present in any product, such as a food product or any therapeutic composition, in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Terpene compounds can be present in any product, such as a food product or any therapeutic composition, in a quantity of from about 50 to about 150 milligrams. Terpene compounds can be present in any product, such as a food product or any therapeutic composition, in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Terpene compounds can be present in any product, such as a food product or any therapeutic composition, in a quantity of at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Terpene compounds can be present in any product, such as a food product or any therapeutic composition, in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

The compositions of the present disclosure can be enriched in cannabinoids compared to hemp oil. For example, a composition can comprise hemp oil and cannabinoids from plant sources such as extracts (e.g., hemp extract) and essential oils. A composition can comprise about 0%, 10%, 20%, 30%, 40%, 50%, 60%, 70%, 80%, 90%, 100%, 200%, 300%, 400%, 500%, 600%, 700%, 800%, 900%, or 1000% greater concentration of cannabinoids compared to hemp oil.

The compositions of the present disclosure can be enriched in cannabidiol compounds compared to hemp oil. For example, a composition can comprise hemp oil and cannabidiol compounds from plant sources such as extracts (e.g., hemp extract) and essential oils. A composition can comprise about 0%, 10%, 20%, 30%, 40%, 50%, 60%, 70%, 80%, 90%, 100%, 200%, 300%, 400%, 500%, 600%, 700%, 800%, 900%, or 1000% greater concentration of cannabidiol compounds compared to hemp oil.

The compositions of the present disclosure can be enriched in THC compounds compared to hemp oil. For example, a composition can comprise hemp oil and THC compounds from plant sources such as extracts (e.g., hemp extract) and essential oils. A composition can comprise about 0%, 10%, 20%, 30%, 40%, 50%, 60%, 70%, 80%, 90%, 100%, 200%, 300%, 400%, 500%, 600%, 700%, 800%, 900%, or 1000% greater concentration of THC compounds compared to hemp oil.

The compositions of the present disclosure can be enriched in terpenes compared to hemp oil. For example, a composition can comprise hemp oil and terpenes from plant sources such as extracts (e.g., hemp extract) and essential oils. A composition can comprise about 0%, 10%, 20%, 30%, 40%, 50%, 60%, 70%, 80%, 90%, 100%, 200%, 300%, 400%, 500%, 600%, 700%, 800%, 900%, or 1000% greater concentration of terpenes compared to hemp oil.

Compounds included in the compositions of the present disclosure can be derived from various sources. Compound sources can be natural, such as plant extracts or essential oils. Compounds in the compositions of the present disclosure can be derived from hemp oil, including cannabinoid compounds, THC compounds, and terpene compounds. Compounds in the compositions of the present disclosure can be derived from essential oils, including but not limited to those essential oils discussed further in this disclosure. These compounds can include cannabinoid compounds and terpene compounds. In some cases, all the compounds or ingredients in a composition are natural or naturally-derived. In some cases, all the compounds or ingredients in a composition are vegetarian. In some cases, all the compounds or ingredients in a composition are vegan.

Terpenes and/or essential oils in compositions of the present disclosure can be selected to provide benefits for particular conditions or subjects. Terpenes and/or essential oils can be employed in combination with each other, as well as in combination with cannabinoids, for example to target treatment of particular conditions. For example, terpinolene, terpineol and linalool or lavender, valerian and jasmine essential oils can be combined with cannabinoids or cannabis extract to act as a sleep aid or treat sleep disorders.

Alpha-pinene can be used as an anti-inflammatory, an antiangiogenic, an anti-ulcer agent, and a bronchodilator.

Linalool can be used for reducing anxiety, reducing inflammation (e.g., lung inflammation), to improve Alzheimer's disease or symptoms thereof, as a sedative, an analgesic, an anti-microbial, an antibacterial, and an anti-epileptic.

Myrcene can be used as an antibacterial, a neuroprotective agent, an antinociceptive, an analgesic, and to alleviate neuropathic pain, peptic ulcer disease, and inflammation. Depending on concentration, myrcene can be used as a sedative (e.g., over 0.5% myrcene) or to provide energizing effects (e.g., less than 0.5% myrcene).

Limonene can be used to reduce anxiety and depression, to dissolve cholesterol-containing gallstones, to neutralize gastric acid, support normal peristalsis, relieve heartburn and gastroesophageal reflux, to improve immune function, and as a chemopreventative against cancer.

Ocimene can be used as an antifungal agent, an antitumor agent, and a cyctotoxic agent.

Terpinolene can be used for antioxidant, mood regulation, central nervous system (CNS) regulation, anti-inflammatory, anti-diarrheal, anti-filarial, anti-fungal, antimalarial, anti-amoebic, anti-bacterial, cytotoxic, and anticancer effects.

Terpineol can be used to relax a subject, to aid digestion and improve gastrointestinal disorders, and to relieve influenza, bronchitis, cough, nasal congestion, and sinusitis.

Beta-caryophyllene can be used as an anti-inflammatory agent, an anti-tumor agent, and an analgesic.

Geraniol can be used to reduce or protect against neuropathy, as an antidepressant, to suppress angiogenesis, to improve anti-cancer agent efficacy, to suppress growth of cancer cells (e.g., lung cancer), as a chemopreventive against cancer, to reduce inflammation and apoptosis (e.g., in liver cells), to reduce oxidative stress, as an antioxidant, and as an antimicrobial.

Alpha-humulene can be used as an appetite suppressant, an anti-inflammatory agent, an insect repellant, an antibacterial, an antioxidant, and an allelopathic agent.

Phellandrene can be used as an antidepressant and an antihyperalgesic.

Carene can be used as an antioxidant, an antiproliferative, an antimicrobial, and to reduce excess body fluid production, such as of tears, mucous, or sweat.

Terpinene can be used as an antioxidant, an anti-inflammatory, an antimicrobial, an antiproliferative, to reduce oxidative stress, and to manage diabetes.

Fenchol can be used as an antibacterial agent, an antimycobacterial, an antimicrobial, and an antioxidant.

Borneol can be used to alleviate hyperalgesia, as a TRPA1 inhibitor, an anti-inflammatory agent, and an anti-nociceptive agent.

Bisabolol can be used as an anti-cancer agent, such as to induce apoptosis in leukemia, an anti-tumor agent (e.g., pancreatic cancer), and an antigenotoxicity agent.

Phytol can be used to relax a subject, such as by inhibiting degradation of GABA, as an anxiolytic, to resist menadione-induced oxidative stress, and as an antimicrobial.

Camphene can be used for pain relief, as an antioxidant, to induce apoptosis in cancer cells (e.g., melanoma), an antitumor agent, and an antibacterial.

Sabinene can be used as an antioxidant, an antimicrobial, an anticancer agent (e.g., oral, liver, lung, colon, melanoma, and leukemic cancer), to aid liver function, aid digestion, relieve arthritis, and relieve skin conditions.

Camphor can be used to improve skin healing (e.g., reconstructed human epidermis), as a local anesthetic, a muscle relaxant, an antipathogenic, and an antimicrobial agent.

Isoborneol can be used as an antioxidant, a cytotoxic, a DNA-protective, to inhibit herpes simplex virus type 1, and to inhibit HIV.

Menthol can be used as an analgesic, to desensitize α3β4 nicotinic acetylcholine receptors, as an antinociceptive, and as an anti-inflammatory agent.

Nerolidol can be used as an antifungal agent, an antimicrobial agent, an antioxidant, and an antimalarial agent.

Guaiol can be used as an antimicrobial agent, an antifungal agent, and an antibiotic.

Isopulegol can be used as a gastroprotective agent, an anti-inflammatory agent, to enhance permeability for transdermal administration of compounds, and to reduce the severity of seizures.

Geranyl acetate can be used as an antimicrobial agent, an antibacterial, and an antioxidant.

Cymene can be used as an anti-inflammatory agent, an anti-hyperalgesic, an antioxidant, an anti-diabetic, to aid in weight loss, to aid immune disorders, and to protect against acute lung injury.

Eucalyptol can be used as an antifungal agent, to alleviate inflammation (e.g., lung inflammation), an antioxidant, and an anticancer agent.

Pulegone can be used to enhance skin permeability, as an insecticide, and an antioxidant.

The compositions of the present disclosure can comprise one or more essential oils or essential oil compounds. Essential oils can include, but are not limited to: Linalool; B-Caryophyllene; B-Myrcene; D-Limonene; Humulene; a-Pinene; Ylang Ylang (Cananga odorata); Yarrow (Achillea millefolium); Violet (Viola odorata); Vetiver (Vetiveria zizanoides); Vanilla (Vanilla plantifolia); Tuberose (Polianthes tuberosa); Thyme (Thymus vulgaris L.); Tea Tree (Melaleuca alternifolia); Tangerine (Citrus reticulata); Spruce, Black (Picea mariana); Spruce (Tsuga Canadensis); Spikenard (Nardostachys jatamansi); Spearmint (Mentha spicata); Sandalwood (Santalum spicatum); Rosewood (Aniba rosaeodora); Rosemary Verbenone (Rosmarinus officinalis); Rosemary (Rosmarinus officinalis); Rose (Rosa damascena); Rose Geranium (Pelargonium roseum); Ravensara (Ravensara aromatica); Plai (Zingiber cassumunar) Pine Needle (Pinus sylvestris L.); Petitgrain (Citrus aurantium); Peppermint (Mentha piperita); Pepper, Black (Piper nigrum L.); Patchouli (Pogostemon cablin); Palo Santo (Bursera graveolens); Palmarosa (Cymbopogon martini); Osmanthus (Osmanthus fragrans); Oregano (Origanum vulgare); Orange, Sweet (Citrus sinensis); Oak Moss (Evernia prunastri); Nutmeg (Myristica fragrans) Niaouli (Melaleuca viridifloria); Neroli (aka Orange Blossom) (Citrus aurantium); Myrtle (Myrtus communis); Myrrh (Commiphora myrrha); Mimosa (Acacia decurrens); Melissa (Melissa officinalis L.); Marjoram, Sweet (Origanum majorana); Manuka (Leptospermum scoparium); Mandarin, Red (Citrus deliciosa); Mandarin (Citrus deliciosa); Lotus, White (Nelumbo nucifera); Lotus, Pink (Nelumbo nucifera); Lotus, Blue (Nelumbo nucifera); Lime (Citrus aurantifolia); Lily (Lilum aurantum); Lemongrass (Cymbopogon citratus); Lemon (Citrus limonum); Lavender (Lavandula angustifolium); Lavandin (Lavandula hybrida grosso); Kanuka (Kunzea ericoides); Juniper Berry (Juniperus cummunis); Jasmine (Jasminum officinale); Jasmine Abs (Jasminum sambac); Helichrysum (Helichrysum italicum); Grapefruit, White (Citrus x paradisi); Grapefruit, Pink (Citrus paradisi); Ginger (Zingiber officinalis); Geranium (Pelargonium graveolens); Geranium, Bourbon (Pelargonium graveolens, 'Herit); Gardenia (Gardenia jasminoides); Galbanum (Ferula galbaniflua); Frankincense (Boswellia carterii); Frangipani (Plumeria alba); Fir Needle White (Abies alba); Fir Needle Siberia (Abies siberica); Fir Needle Canada (Abies balsamea); Fennel, Sweet (Foeniculum vulgare); Eucalyptus Smithii. Eucalyptus Radiata, Eucalyptus Globulus, Eucalyptus Citriodora, Eucalyptus Blue Mallee (Eucalyptus polybractea); Elemi (Canarium luzonicum); Dill (Anethum graveolens); Cypress (Cupressus sempervirens); Cumin (Cuminum cyminum); Coriander (Coriandum sativum); Cocoa (Theobroma cacao); Clove (Eugenia caryophylatta); Clary Sage (Salvia sclarea); Cistus (aka Labdanum) (Cistus ladaniferus L.); Cinnamon (Cinnamomum zeylanicum); Chamomile, Roman (Anthemis nobilis); Chamomile, Blue (Matricaria chamomilla); Celery Seed (Apium graveolins); Cedarwood, Western Red (Thuja plicata); Cedarwood, Blood (Juniperus virginiana); Cedarwood Atlas (Cedrus atlantica); Carrot Seed (Daucus carota); Cardamon (Elettaria cardamomum); Caraway Seed (Carum carvi); Cajeput (Melaleuca cajuputi); Cade (Juniperus oxycedrus); Birch, White (Betula alba); Birch, Sweet (Betula lenta); Bergamot (Citrus bergamia); Bay Laurel (Laurus nobilis); Basil (Ocimum basilicum); Basil, Holy (Ocimum sanctum); Basil (Ocimum basilicum); Balsam Poplar (Populus balsamifera); Balsam Peru (Myroxylon balsamum); Angelica (Angelica archangelica L.); and combinations thereof.

The compositions of the present disclosure can comprise one or more therapeutic compounds, including but not limited to hydroxychloroquine, chroloquine, azithromycin, dexamethasone, niacin, steroids (e.g., corticosteroids, etc.), vitamins (e.g., vitamin C, vitamin D, vitamin B, etc.), stem cells, baloxavir, chloroquine phosphate, lopinavir, ritonavir, neuraminidase inhibitors (e.g., oseltamivir), remedesivir, ascorbic acid, methylprednisolone, nitric oxide, sarilumab, sirolimus, tocilizumab, angiotensin converting enzyme (ACE) inhibitors, angiotensin II receptor blockers (ARBs), ibuprofen, indomethacin, and niclosamide.

The compositions of the present disclosure can comprise one or more additional ingredients, including but not limited to mushrooms or mushroom derivative products (e.g., reishi mushroom, chaga mushroom, maitake mushroom, oyster mushroom, cordyceps), maca (Lepidium meyenii), he sho wu (also he show wu or shou wu chih), superfoods or superfood derivative products (e.g., blueberries, acai berries, inca berries, goji berries, camucamu, coconut, lucuma, kale, cacao (e.g., cacao powder, cacao butter), sacha inchi, chia, flax, hemp, amaranth, quinoa, moringa oleifera), and combinations thereof.

Compounds used in compositions of the present disclosure can be extracted by a variety of methods. For example, extraction can be performed by maceration, infusion, decoction, percolation, Soxhlet extraction, pressurized solvent extraction, counter current extraction, ultrasonication, or supercritical fluid (e.g., carbon dioxide) extraction.

In some cases, compounds used in compositions of the present disclosure are extracted via supercritical fluid (e.g., carbon dioxide) extraction. For example, cannabinoid compounds can be extracted from hemp (e.g., hemp stalk and hemp stems) using supercritical carbon dioxide extraction.

The compositions of the present disclosure can comprise pregnenolone, including derivatives thereof. Pregnenolone can help protect a subject from cannabis intoxication, for example from THC. Pregnenolone or derivatives thereof can be formulated to be water soluble. A composition of the present disclosure can comprise between about 1 and 50 milligrams (mg) of pregnenolone or derivatives thereof. For example, a unit dosage of the present disclosure can comprise between about 1 and 50 milligrams (mg) of pregnenolone. Compositions of the present disclosure (e.g., unit dosages) can comprise about 1, 2, 3, 4, 5, 6, 7, 8, 9, 10, 15, 20, 25, 30, 35, 40, 45, or 50 mg of pregnenolone. Compositions of the present disclosure (e.g., unit dosages) can comprise at least about 1, 2, 3, 4, 5, 6, 7, 8, 9, 10, 15, 20, 25, 30, 35, 40, 45, or 50 mg of pregnenolone. Compositions of the present disclosure (e.g., unit dosages) can comprise at most about 1, 2, 3, 4, 5, 6, 7, 8, 9, 10, 15, 20, 25, 30, 35, 40, 45, or 50 mg of pregnenolone. Compositions comprising pregnenolone can be used in combination with any other compounds, ingredients, or formulations described herein, including esters, cyclodextrin complexes, microcapsules (e.g., sodium alginate microcapsules), immediate release formulations, delayed or extended release formulations, transbuccal formulations, and sublingual formulations.

Compositions of the present disclosure can be used to treat various diseases or conditions in subjects (e.g., humans, mammals, vertebrates), including but not limited to ALS, Alzheimer's, antibacterial resistant infections, anxiety, atherosclerosis, arthritis, asthma, cancer, colitis, Crohn's, diabetes, depression, endocrine disorders, epilepsy, seizures, fibromyalgia, glaucoma, heart disease, Huntington's, inflammation, irritable bowel syndrome (IBS), kidney disease, liver disease, motion sickness, nausea, neurodegeneration, neuropathic pain, neuropathy, obesity, obsessive compulsive disorder (OCD), osteoporosis, Parkinson's, prion diseases, Mad Cow disease, post-traumatic stress disorder (PTSD), rheumatism, schizophrenia, sickle cell anemia, skin conditions (e.g., psoriasis, dermatitis, allergic inflammation, chronic pruritus), sleep disorders (e.g., sleep-wake disorders, apnea), spinal cord injury, stress, stroke, traumatic brain injury (TBI), and virus-induced diseases such as the COVID-19 or diseases caused by any of the viruses described herein. The compositions of the present disclosure can be provided as a dry powder. For example, an oil-based composition (e.g., hemp oil) can be combined with a drying or powdering agent, such as cyclodextrin. In some cases, a powder composition can be provided on its own. In other cases, a powder composition can be provided in another product, such as a food product, cosmetic product, or other products and compositions such as those disclosed herein.

The compositions of the present disclosure can be provided in any suitable form, including but not limited to a liquid form, a gel form, a semi-liquid (e.g., a liquid, such as a viscous liquid, containing some solid) form, a semi-solid (a solid containing some liquid) form, or a solid form. Compositions can be provided in, for example, a tablet form, a capsule form, a food form a chewable form, a non-chewable form, a transbuccal form, a sublingual form, a slow-release form, a non-slow-release form, a sustained release form, or a non-sustained-release form.

The compositions of the present disclosure can be administered in any oral dosage form, including liquid dosage forms (e.g., a suspension or slurry), and oral solid dosage forms (e.g., a tablet or bulk powder). Tablets can include tablets, caplets, capsules, including soft gelatin capsules, and lozenges. Tablets can further comprise suitable binders, lubricants, diluents, disintegrating agents, colorants, flavoring agents, flow-inducing agents, and melting agents. The compositions of the present disclosure can be administered through the nasal tract.

The compositions of the present disclosure can be administered transdermally, such as via a patch. The compositions of the present disclosure can be administered intravenously. The compositions of the present disclosure can be administered topically. The compositions of the present disclosure can be administered via exposure to an aqueous solution, such as a subject immersing in a float tank. The compositions of the present disclosure can be formulated as a bath salt or liquid bath product, which can be dissolved or dispersed in water (e.g., a bath) for skin exposure, for example by immersion of the subject.

The compositions of the present disclosure can be provided as cosmetics or personal care products, such as soaps (e.g., solid, bar, liquid, or foaming), hand sanitizer, lotions, massage oils masks, makeup, moisturizers, sunscreen, toothpaste, mouth wash, or throat spray. Use of cannabinoids in such applications can provide benefits including reduction of inflammation in a subject.

The compositions of the present disclosure can be provided as a food composition in combination with a food carrier, including but not limited to food bars (e.g., granola bars, protein bars, candy bars), cereal products (e.g., oatmeal, breakfast cereals, granola), bakery products (e.g., bread, donuts, crackers, bagels, pastries, cakes), dairy products (e.g., milk, yogurt, cheese), beverages (e.g., milk-based beverages, sports drinks, fruit juices, teas, soft drinks, alcoholic beverages, bottled waters), beverage mixes, pastas, grains (e.g., rice, corn, oats, rye, wheat, flour), egg products, snacks (e.g., candy, chips, gum, gummies, lozenges, mints, chocolate), meats, fruits, vegetables or combinations thereof. Food compositions can comprise solid foods. Food compositions can comprise semi-solid foods. Food compositions can comprise liquid foods. A composition in a liquid form may be formulated from a dry mix, such as a dry beverage mix or a powder. A dry mix may be suitable in terms of transportation, storage, or shelf life. The composition can be formulated from the dry mix in any suitable manner, such as by adding a suitable liquid (e.g., water, milk, fruit juice, tea, or alcohol).

A food composition or food product can comprise a food bar, including but not limited to granola bars, protein bars, candy bars, and energy bars. A food composition or food product can comprise a cereal product, including but not limited to oatmeal, flour (e.g., wheat flour, rice flour, corn flour, barley flour), breakfast cereal, granola, bread, pasta, rice cakes, and popcorn. A food composition or food product can comprise a bakery product, including but not limited to bread, pastries, brownies, cakes, pies, donuts, crackers, and muffins. A food composition or food product can comprise a dairy product, including but not limited to milk, fermented milk, curd, whey, yogurt, cream, cheese, butter, clarified butter, ghee, and ice cream. A food composition or food product can comprise a nut butter or seed butter, including but not limited to peanut butter, almond butter, cashew butter, hazelnut butter, macadamia nut butter, pecan butter, pistachio butter, walnut butter, pumpkin seed butter, sesame seed butter, soybean butter, and sunflower seed butter. A food composition or food product can comprise an oil (e.g., a cooking oil), including but not limited to olive oil, coconut oil, vegetable oil, canola oil, corn oil, peanut oil, sunflower seed oil, almond oil, avocado oil, rice bran oil, cottonseed oil, flaxseed oil, linseed oil, grape seed oil, hemp oil, mustard oil, macadamia oil, palm oil, tea seed oil, walnut oil, margarine, lard, butter, clarified butter, ghee, or tallow. A food composition or food product can comprise sports food products such as energy gels, sports drinks, energy powders, energy bars, energy shots, protein powders, and protein drinks (e.g., protein shakes). A food composition or food product can comprise a beverage, including but not limited to water, electrolyte drinks, soda, coconut water, tea (e.g., Jun tea, black tea, green tea, white tea, herbal tea), coffee, a soft drink, an alcoholic beverage (e.g., cocktail, liquor, spirits, beer, wine, malt beverage), water, juice (e.g., apple juice, orange juice, tomato juice, vegetable juice, cranberry juice), a sports drink, electrolyte-enriched water, vitamin-enhanced water, a hangover-recovery drink, milk (e.g., dairy-based milk, coconut milk, almond milk, soy milk, hemp milk, rice milk, oat milk, cashew milk, hazelnut milk), and yogurt. A food composition or food product can comprise a fungus or fermented food or drink, including but not limited to kifir (kefir), jun, amasi, amazake, appam, ayran, doogh, bagoong, brem, cheonggukjang, chicha, kombucha, fermented bean curd, kimchi, lassi, miso, poi, yakult, and yogurt.

Compositions of the present disclosure can comprise pet or other animal products, such as animal food (e.g., dog food, cat food), treats, and nutritional supplements (e.g., liquids, sprays, or powders for application to food or water). These compositions can be formulated for or administered to domestic or pet animals (e.g., dogs, cats, small mammals, birds), livestock and other farm animals (e.g., cows, pigs, horses, sheep, goats), zoo animals, or any other vertebrates. Compositions for administration to animals can be formulated with microencapsulated cannabinoid-rich oil or non-encapsulated cannabinoid-rich oil, alone or in combination with essential oils, terpenes, and other components described herein. Compositions for administration to animals can be mixed into feed or water, prepared for spraying application (e.g., mixed in glycerin), for intravenous administration (e.g., in a syringe or an IV bag), in salves, vitamins, liquid vitamin pumps, treats, or other forms.

The compositions of the present disclosure can comprise an additional agent or agents, whether active or passive. Examples of such an agent include a sweetening agent, a flavoring agent, a coloring agent, a filling agent, a binding agent, a lubricating agent, an excipient, a preservative, or a manufacturing agent. Additional pharmaceutically acceptable excipients (in the case of pharmaceuticals) or other additives (for non-pharmaceutical applications) can be added to the composition. For example, if desired, any generally accepted soluble or insoluble inert pharmaceutical filler (diluent) material can be included in the final product (e.g., a solid dosage form). Such inert pharmaceutical filler can comprise a monosaccharide, a disaccharide, a polyhydric alcohol, inorganic phosphates, sulfates or carbonates, and combinations thereof. Examples of suitable inert pharmaceutical fillers include sucrose, dextrose, lactose, xylitol, fructose, sorbitol, calcium phosphate, calcium sulfate, calcium carbonate, microcrystalline cellulose, and combinations thereof. An effective amount of any generally accepted pharmaceutical lubricant, such as calcium or magnesium soaps, can be added.

The compositions of the present disclosure can be administered to a subject. Compositions can be administered in a variety of ways, including but not limited to oral and topical administration.

Administering the compositions of the present disclosure to a subject can provide one or more beneficial effects. Beneficial effects can include but are not limited to pain relief, reduced bacterial growth, reduced blood sugar levels, improved blood lipid and cholesterol profiles, increased fat burning, reduced appetite, stimulated appetite, reduced vomiting or nausea, reduced seizures or convulsions, antifungal effects, reduced inflammation, reduced arthritis (e.g., rheumatoid arthritis), reduced insomnia or aided sleep, reduced arterial blockage, inhibited cancer cell growth, improved psoriasis, tranquilizing effects, antispasmodic effects, reduced anxiety, bone growth promotion, reduced intestinal contractions, and nervous system protection.

Any of the subject compositions can be provided in a unit dosage form. A unit dosage is an amount of a compound, such as a cannabinoid compound delivered alone or in combination with other components, which is to be administered to a subject at or about one time point. Other components which can be included with a unit dosage include but are not limited to cosmetics, food carriers, food bars, baked goods, dairy products, oils, beverages, solid dosages (e.g., tablets), or liquid dosages. A unit dosage of a cannabinoid compound can be about 1, 2, 3, 4, 5, 6, 7, 8, 9, 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage of a cannabinoid compound can be at least about 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage of a cannabinoid compound can be at most about 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage of a non-cannabinoid therapeutic compound can be about 1, 2, 3, 4, 5, 6, 7, 8, 9, 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage of a non-cannabinoid therapeutic compound can be at least about 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage of a non-cannabinoid therapeutic compound can be at most about 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage of a therapeutic compound can be about 1, 2, 3, 4, 5, 6, 7, 8, 9, 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage of a therapeutic compound can be at least about 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage of a therapeutic compound can be at most about 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage can be an hourly dosage. A unit dosage can be a daily dosage. A unit dosage can provide about 1/24, 1/12, 1/8, 1/6, 1/4, 1/3, 1/2, or all of a daily dosage of one or more cannabinoids for a subject. A unit dosage can take the form of a tablet, gel, liquid, food product, food bar, container of liquid of defined volume, or other forms described herein, packaged for one-time consumption or administration.

The compositions described herein can provide several advantages, when administered to a subject, including but not limited to at least one of (e.g., one, two, or all of) (i) reduced expression or activity of a cytokine in a target cell, when the administering occurs to the subject having a viral infection, (ii) reduced platelet activation, and (iii) reduced expression or activity of angiotensin pathway protein in a target cell, as compared to a corresponding control.

In some cases, the corresponding control is a control composition comprising (i) at least one cannabinoid compound in non-encapsulated form and/or (ii) at least one non-cannabinoid therapeutic compound in non-encapsulated form.

The compositions described herein, when administered to a subject, can effect reduced expression and/or activity of at least one cytokine in at least one target cell. Upon administration of the composition(s), a target cell can exhibit reduced expression and/or activity at least 1, 2, 3, 4, 5, 6, 7, 8, 9, 10, or more different cytokines, and/or respective receptor(s) thereof. For example, upon administration of the composition(s), the target cell can exhibit reduced cytokine storm. Upon administration of the composition(s), the reduced expression and/or activity of the cytokine(s) in the target cell can persist for at least about 0.1, 0.2, 0.5, 1, 2, 4, 6, 12, 18, 24 hour(s), 2 days, 3 days, 4 days, 5 days, 6 days, 7 days, 2 weeks, 3 weeks, 4 weeks, or 2 months. Upon administration of the composition(s), the reduced expression and/or activity of the cytokine(s) in the target cell can persist for about 0.1, 0.2, 0.5, 1, 2, 4, 6, 12, 18, 24 hour(s), 2 days, 3 days, 4 days, 5 days, 6 days, 7 days, 2 weeks, 3 weeks, 4 weeks, or 2 months. Upon administration of the composition(s), the reduced expression and/or activity of the cytokine(s) in the target cell can persist for at most about 0.1, 0.2, 0.5, 1, 2, 4, 6, 12, 18, 24 hour(s), 2 days, 3 days, 4 days, 5 days, 6 days, 7 days, 2 weeks, 3 weeks, 4 weeks, or 2 months. The composition(s) as described herein can be characterized by reduced expression and/or activity level of the cytokine(s) in the target cell, which level is at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than expression and/or activity level of the cytokine(s) upon treatment with a corresponding control. The composition(s) as described herein can be characterized by reduced expression and/or activity of the cytokine(s) in the target cell, which level is about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than expression and/or activity level of the cytokine(s) upon treatment with a corresponding control. The composition(s) as described herein can be characterized by reduced expression and/or activity of the cytokine(s) in the target cell, which level is at most about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than expression and/or activity level of the cytokine(s) upon treatment with a corresponding control.

Cytokines as disclosed herein can comprise one or more members selected from interferons (IFN), interleukins (IL), chemokines, colony-stimulating factors (CSF), and/or tumor necrosis factor (TNF). Non-limiting examples of IFN can include type I IFN (IFN-alpha and IFN-beta), type II IFN (IFN-gamma), and type II IFN (IFN-gammal, IFN- gamma2, IFN-gamma3)). Non-limiting examples of IL can include IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, and IL-38. Non-limiting examples of chemokines can include CXCL1, CXCL2, CXCL8, CXCL9, CXCL10, CCL2, CCL20, etc. Non-limiting examples of CSF can include granulocyte-macrophage colony-stimulating factor (GM-CSF), macrophage colony-stimulating factor (M-CSF), and granulocyte colony-stimulating factor (G-CSF). Non-limiting examples of TNF can include TNF-alpha.

A target cell (e.g., exhibiting reduced expression and/or activity of at least one cytokine), as disclosed herein, can be an immune cell. An immune cell can comprise one or more lymphoid cells, such as B cell, T cell (e.g., Cytotoxic T cell, Natural Killer T cell, Regulatory T cell, T helper cell), Natural killer cell, cytokine induced killer (CIK) cells, etc. Alternatively or in addition to, an immune cell can comprise one or more myeloid cells, such as granulocytes (e.g., Basophil granulocyte, Eosinophil granulocyte, Neutrophil granulocyte, Hypersegmented neutrophil), Monocyte/Macrophage, Red blood cell (e.g., Reticulocyte), Mast cell, Thrombocyte, Megakaryocyte, Dendritic cell, etc.

The compositions described herein, when administered to a subject, can effect reduced platelet activation. The reduced platelet activation can effect or can be a sign of (i) treatment or (ii) reduced occurrence of thrombosis and thrombosis related diseases/disorders. The reduced platelet activation can effect or can be a sign of reduced blood clot formation in (i) a blood vessel of the subject or (ii) a blood sample derived from the subject. The composition(s) as described herein can be characterized by reduced platelet activation level in the subject or a blood sample derived from the subject, which level is at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than platelet activation level upon treatment with a corresponding control. The composition(s) as described herein can be characterized by reduced platelet activation level in the subject or a blood sample derived from the subject, which level is about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than platelet activation level upon treatment with a corresponding control. The composition(s) as described herein can be characterized by reduced platelet activation level in the subject or a blood sample derived from the subject, which level is at most about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than platelet activation level upon treatment with a corresponding control.

The compositions described herein, when administered to a subject, can effect reduced expression and/or activity level of a chemokine in a platelet. Non-limiting examples of the chemokine can include, but are not limited to, CXCL1, CXCL4, CXCLS, CXCL7, CXCL8, CXCL12, CCL3, and CCLS. The composition(s) as described herein can be characterized by reduced chemokine expression and/or activity level in the subject or a blood sample derived from the subject, which level is at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than chemokine expression and/or activity level upon treatment with a corresponding control. The composition(s) as described herein can be characterized by reduced chemokine expression and/or activity level in the subject or a blood sample derived from the subject, which level is about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than chemokine expression and/or activity level upon treatment with a corresponding control. The composition(s) as described herein can be characterized by reduced chemokine expression and/or activity level in the subject or a blood sample derived from the subject, which level is at most about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than chemokine expression and/or activity level upon treatment with a corresponding control.

The compositions described herein, when administered to a subject, can effect reduced expression and/or activity level of a cell-adhesion molecule in a platelet. Non-limiting examples of the cell-adhesion molecule can include, but are not limited to, glycoprotein P-selectin, and integrin. The composition(s) as described herein can be characterized by reduced cell-adhesion molecule expression and/or activity level in the subject or a blood sample derived from the subject, which level is at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than cell-adhesion molecule expression and/or activity level upon treatment with a corresponding control. The composition(s) as described herein can be characterized by reduced cell-adhesion molecule expression and/or activity level in the subject or a blood sample derived from the subject, which level is about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than cell-adhesion molecule expression and/or activity level upon treatment with a corresponding control. The composition(s) as described herein can be characterized by reduced cell-adhesion molecule expression and/or activity level in the subject or a blood sample derived from the subject, which level is at most about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than cell-adhesion molecule expression and/or activity level upon treatment with a corresponding control.

The compositions described herein, when administered to a subject, can effect reduced expression and/or release level of hemostatically active molecule(s) (e.g., adenosine diphosphate (ADP), adenosine triphosphate (ATP), calcium, catecholamines such as serotonin and histamine, etc.) in a platelet. The composition(s) as described herein can be characterized by reduced hemostatically active molecule(s) expression and/or release level in the subject or a blood sample derived from the subject, which level is at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than hemostatically active molecule(s) expression and/or release level upon treatment with a corresponding control. The composition(s) as described herein can be characterized by reduced hemostatically active molecule(s) expression and/or release level in the subject or a blood sample derived from the subject, which level is about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than hemostatically active molecule(s) expression and/or release level upon treatment with a corresponding control. The composition(s) as described herein can be characterized by reduced hemostatically active molecule(s) expression and/or release level in the subject or a blood sample derived from the subject, which level is at most about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than hemostatically active molecule(s) expression and/or release level upon treatment with a corresponding control.

The compositions described herein, when administered to a subject, can effect reduction in platelet aggregation. The composition(s) as described herein can be characterized by reduced platelet aggregation level in the subject or a blood sample derived from the subject, which level is at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than platelet aggregation level upon treatment with a corresponding control. The composition(s) as described herein can be characterized by reduced platelet aggregation level in the subject or a blood sample derived from the subject, which level is about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than platelet aggregation level upon treatment with a corresponding control. The composition(s) as described herein can be characterized by reduced platelet aggregation level in the subject or a blood sample derived from the subject, which level is at most about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than platelet aggregation level upon treatment with a corresponding control. Platelet aggregation level as disclosed herein can be measured by, for example, light-transmission aggregometry (LTA) assay or whole blood aggregometry (WBA) assay.

The compositions described herein, when administered to a subject, can effect reduced expression and/or activity of at least one angiotensin pathway protein (or renin-angiotensin system (RAS) protein, as used interchangeably herein) in at least one target cell. Upon administration of the composition(s), a target cell can exhibit reduced expression and/or activity at least 1, 2, 3, 4, 5, 6, 7, 8, 9, 10, or more different angiotensin pathway proteins. Upon administration of the composition(s), the reduced expression and/or activity of the angiotensin pathway protein(s) in the target cell can persist for at least about 0.1, 0.2, 0.5, 1, 2, 4, 6, 12, 18, 24 hour(s), 2 days, 3 days, 4 days, 5 days, 6 days, 7 days, 2 weeks, 3 weeks, 4 weeks, or 2 months. Upon administration of the composition(s), the reduced expression and/or activity of the angiotensin pathway protein(s) in the target cell can persist for about 0.1, 0.2, 0.5, 1, 2, 4, 6, 12, 18, 24 hour(s), 2 days, 3 days, 4 days, 5 days, 6 days, 7 days, 2 weeks, 3 weeks, 4 weeks, or 2 months. Upon administration of the composition(s), the reduced expression and/or activity of the angiotensin pathway protein(s) in the target cell can persist for at most about 0.1, 0.2, 0.5, 1, 2, 4, 6, 12, 18, 24 hour(s), 2 days, 3 days, 4 days, 5 days, 6 days, 7 days, 2 weeks, 3 weeks, 4 weeks, or 2 months. The composition(s) as described herein can be characterized by reduced expression and/or activity level of the angiotensin pathway protein(s) in the target cell, which level is at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than expression and/or activity level of the angiotensin pathway protein(s) upon treatment with a corresponding control. The composition(s) as described herein can be characterized by reduced expression and/or activity of the angiotensin pathway protein(s) in the target cell, which level is about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than expression and/or activity level of the angiotensin pathway protein(s) upon treatment with a corresponding control. The composition(s) as described herein can be characterized by reduced expression and/or activity of the angiotensin pathway protein(s) in the target cell, which level is at most about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than expression and/or activity level of the angiotensin pathway protein(s) upon treatment with a corresponding control.

Non-limiting examples of the angiotensin pathway protein can include angiotensin (e.g., angiotensin I, angiotensin II), angiotensin receptor (e.g., angiotensin I receptor (AT1), angiotensin II receptor (AT2)), angiotensin-converting enzyme (ACE), angiotensinogen, renin, derivatives thereof, functional variants thereof, and combinations thereof.

A target cell (e.g., exhibiting reduced expression and/or activity of at least one angiotensin pathway protein), as disclosed herein, can be a cell found in or derived from one or more biological tissues, such as, for example, endocrine tissues, gastrointestinal tract (e.g. ileum, liver and gallbladder), cardiovascular tissues, kidney and urinary bladder, testes, and muscle tissues. Non-limiting examples of such target cell can include vascular endothelial cells, epithelial kidney cells, and testicular Leydig cells, sperm cells, etc.

The compositions described herein, when administered to a subject, can effect reduced viral infection in a target cell of the subject. Upon administration of the composition(s), the target cell can exhibit reduced viral infection which can persist for at least about 0.1, 0.2, 0.5, 1, 2, 4, 6, 12, 18, 24 hour(s), 2 days, 3 days, 4 days, 5 days, 6 days, 7 days, 2 weeks, 3 weeks, 4 weeks, or 2 months. Upon administration of the composition(s), the reduced viral infection in the target cell can persist for about 0.1, 0.2, 0.5, 1, 2, 4, 6, 12, 18, 24 hour(s), 2 days, 3 days, 4 days, 5 days, 6 days, 7 days, 2 weeks, 3 weeks, 4 weeks, or 2 months. Upon administration of the composition(s), the reduced viral infection in the target cell can persist for at most about 0.1, 0.2, 0.5, 1, 2, 4, 6, 12, 18, 24 hour(s), 2 days, 3 days, 4 days, 5 days, 6 days, 7 days, 2 weeks, 3 weeks, 4 weeks, or 2 months. The composition(s) as described herein can be characterized by reduced viral infection level in the target cell, which level is at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than viral infection level in a cell upon treatment with a corresponding control. The composition(s) as described herein can be characterized by reduced viral infection level in the target cell, which level is about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than viral infection level in a cell upon treatment with a corresponding control. The composition(s) as described herein can be characterized by reduced viral infection level in the target cell, which level is at most about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than viral infection level in a cell upon treatment with a corresponding control.

As disclosed herein, a viral infection can refer to any stage of an infection by a virus in a host (e.g., a cell, an animal, a human, etc.), including, but not limited to, attachment of the virus to a cell, penetration of the virus to the cell (e.g., fusion of the virus to the cell membrane, uncoating of the virus), uncoating of capsid of the virus, replication (e.g., transcription or reverse transcription of the viral genome, translation of the viral genome or a derivative thereof, viral particle assembly), and lysis (e.g., release of new viral particles from the cell). Viral infection can also refer to any period of viral infection, including, but not limited to incubation phase, latent phase, dormant phase, acute phase, and development and maintenance of immunity towards a virus.

The compositions described herein (e.g., comprising CBD and/or a derivative thereof, such as, 7-OH-CBD), when administered to a subject, can effect reduced replication of a virus (e.g., coronavirus, such as SARS-CoV-2) in a target cell (e.g., epithelial cells, such as lung epithelial cells) of the subject. Upon administration of the composition(s), the target cell can exhibit reduced replication of the virus which can persist for at least about 0.1, 0.2, 0.5, 1, 2, 4, 6, 12, 18, 24 hour(s), 2 days, 3 days, 4 days, 5 days, 6 days, 7 days, 2 weeks, 3 weeks, 4 weeks, or 2 months. Upon administration of the composition(s), the reduced viral replication in the target cell can persist for about 0.1, 0.2, 0.5, 1, 2, 4, 6, 12, 18, 24 hour(s), 2 days, 3 days, 4 days, 5 days, 6 days, 7 days, 2 weeks, 3 weeks, 4 weeks, or 2 months. Upon administration of the composition(s), the reduced viral replication in the target cell can persist for at most about 0.1, 0.2, 0.5, 1, 2, 4, 6, 12, 18, 24 hour(s), 2 days, 3 days, 4 days, 5 days, 6 days, 7 days, 2 weeks, 3 weeks, 4 weeks, or 2 months. The composition(s) as described herein can be characterized by reduced replication level of the virus in the target cell, which level is at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than replication level of the virus in a cell upon treatment with a corresponding control. The composition(s) as described herein can be characterized by reduced replication level of the virus in the target cell, which level is about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than replication level of the virus in a cell upon treatment with a corresponding control. The composition(s) as described herein can be characterized by reduced replication level of the virus in the target cell, which level is at most about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% lower than replication level of the virus in a cell upon treatment with a corresponding control. A replication level of a virus in a cell, as disclosed herein, may be ascertained by measuring an amount of a viral protein (e.g., spike proteins, capsid proteins, viral envelope proteins, viral membrane fusion proteins, viral non-structural proteins, viral regulatory proteins, viral accessory proteins, etc.) or a viral nucleic acid or a derivative thereof) in a single cell or a population of cells derived from a subject (e.g., derived from a biological tissue from the subject).

As disclosed herein, expression and/or activity level of a protein of interest (e.g., cytokine, chemokine, cell-adhesion molecule, ACE, angiotensin receptor, viral protein(s), etc.) can be measured via, for example, Western blotting. polymerase chain reaction (PCR) techniques, various sequencing methods, and/or enzyme-linked immunosorbent assay (ELISA) assay. Expression and/or activity level of a molecule of interest (e.g., hemostatically active molecule(s)) can be measured, via, for example, any commercially available kits (e.g., colorimetric and/or fluorometric kits, ELISA assay, etc.).

The compositions described herein can provide several advantages, including but not limited to increased shelf stability, increased bioavailability, increased bioactivity, and delayed release. The compositions described herein, when administered to a subject, can have various release profiles, half-lives, and metabolic characteristics. The subject compositions can comprise a plurality of microcapsules, wherein an individual microcapsule in the plurality can be characterized by exhibiting at least one of: (a) a sigmoidal release profile of the at least one cannabinoid compound; (b) a plasma half-life of the at least one cannabinoid compound greater than twice that of the at least one cannabinoid compound in non-encapsulated form; (c) a first pass metabolism of the at least one cannabinoid compound reduced by at least 50% compared to the at least one cannabinoid compound in non-encapsulated form; d) a rate of excretion of the at least one cannabinoid compound from a subject's body reduced by at least 20% compared to the at least one cannabinoid compound in non-encapsulated form; or (e) a degradation rate at an ambient temperature of at least 20° C. of the at least one cannabinoid compound of less than about 50% of a degradation rate of the at least one cannabinoid compound in non-encapsulated form. Alternatively or in addition to, an individual microcapsule in the plurality can be characterized by exhibiting at least one of: (a) a sigmoidal release profile of the at least one non-cannabinoid therapeutic compound; (b) a plasma half-life of the at least one non-cannabinoid therapeutic compound greater than twice that of the at least one non-cannabinoid therapeutic compound in non-encapsulated form; (c) a first pass metabolism of the at least one non-cannabinoid therapeutic compound reduced by at least 50% compared to the at least one non-cannabinoid therapeutic compound in non-encapsulated form; d) a rate of excretion of the at least one non-cannabinoid therapeutic compound from a subject's body reduced by at least 20% compared to the at least one non-cannabinoid therapeutic compound in non-encapsulated form;

or (e) a degradation rate at an ambient temperature of at least 20 ° C. of the at least one non-cannabinoid therapeutic compound of less than about 50% of a degradation rate of the at least one non-cannabinoid therapeutic compound in non-encapsulated form.

The compositions described herein can have a shelf half-life of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 240, 270, 300, 330, or 360 days. In some cases, the compositions described herein can have a shelf half-life of at least about 1, 2, 3, 4, or 5 years. Compositions in microencapsulated form can be characterized by a cannabinoid degradation rate at an ambient temperature of at least 20 ° C. of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than the degradation rate of a non-encapsulated cannabinoid composition. Alternatively or in addition to, compositions in microencapsulated form can be characterized by a non-cannabinoid compound degradation rate at an ambient temperature of at least 20 ° C. of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than the degradation rate of a non-encapsulated non-cannabinoid compound composition.

Cannabinoid compositions in microencapsulated form can be characterized by a plasma half-life in a subject of at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, or 5.0 times that of a non-encapsulated cannabinoid composition. Alternatively or in addition to, non-cannabinoid compositions in microencapsulated form can be characterized by a plasma half-life in a subject of at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, or 5.0 times that of a non-encapsulated non-cannabinoid compound composition. Plasma half-life of a composition can be determined experimentally by administering the composition to a subject, taking plasma samples from a subject at multiple time points, and measuring the concentration of the compound or compounds of interest in those plasma samples. The concentration of the compound or compounds of interest will reach a peak value in the plasma, then fall as the compound or compounds are metabolized, degraded, or cleared from the blood stream. The plasma half-life is the time for the plasma concentration value to be halved.

The cannabinoid release profile can be sigmoidal (e.g., having an ‘S’ shape curve, such as a logistic function). The cannabinoid release profile can be non-sigmoidal. The cannabinoid release profile can be linear. The cannabinoid release profile can be non-linear. The cannabinoid release profile can be instant release. The cannabinoid release profile can be non-instant release. The cannabinoid release profile can be delayed release. The cannabinoid release profile can be constant or sustained release. The cannabinoid release profile can be non-constant or non-sustained release.

The non-cannabinoid compound release profile can be sigmoidal (e.g., having an ‘S’ shape curve, such as a logistic function). The non-cannabinoid compound release profile can be non-sigmoidal. The non-cannabinoid compound release profile can be linear. The non-cannabinoid compound release profile can be non-linear. The non-cannabinoid compound release profile can be instant release. The non-cannabinoid compound release profile can be non-instant release. The non-cannabinoid compound release profile can be delayed release. The non-cannabinoid compound release profile can be constant or sustained release. The non-cannabinoid compound release profile can be non-constant or non-sustained release.

Tablets can be formulated in sustained release format. Methods of making sustained release tablets are known in the art; see, for example, U.S. Patent Publication No. 2006/0051416 and U.S. Patent Publication No. 2007/0065512. Gradual-release tablets are known in the art; examples of such tablets are set forth in U.S. Pat. No. 3,456,049, for example. A slow- or sustained-release form may delay disintegration or absorption of the composition or one or more components thereof.

In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a cannabinoid compound is released from a microcapsule within 1 hour of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a cannabinoid compound is released from a microcapsule within 2 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a cannabinoid compound is released from a microcapsule within 3 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a cannabinoid compound is released from a microcapsule within 4 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a cannabinoid compound is released from a microcapsule within 5 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a cannabinoid compound is released from a microcapsule within 6 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a cannabinoid compound is released from a microcapsule within 7 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a cannabinoid compound is released from a microcapsule within 8 hours of administration to a subject.

In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a non-cannabinoid compound is released from a microcapsule within 1 hour of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a non-cannabinoid compound is released from a microcapsule within 2 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a non-cannabinoid compound is released from a microcapsule within 3 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a non-cannabinoid compound is released from a microcapsule within 4 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a non-cannabinoid compound is released from a microcapsule within 5 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a non-cannabinoid compound is released from a microcapsule within 6 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a non-cannabinoid compound is released from a microcapsule within 7 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a non-cannabinoid compound is released from a microcapsule within 8 hours of administration to a subject.

A release profile is the relationship between time and the amount of a compound released into a subject or the concentration of the compound within the subject (e.g., within the plasma). Release profiles can be measured in a similar manner to plasma half-life. A composition can be administered to a subject, and samples (e.g., plasma samples or blood samples) can be taken from the subject at multiple time points. The concentration of the compound or compounds of interest can be measured in those samples, and a release profile can be plotted.

Compounds taken up into a subject via the gastrointestinal system can be transported to the liver before entering general circulation. Compounds susceptible to metabolic degradation in the liver can have their activities substantially reduced by the first-pass metabolism through the liver. Encapsulation (e.g., microencapsulation) of compounds can reduce first-pass metabolism of the compounds in the liver. Compositions in microencapsulated form can be characterized by a first pass cannabinoid metabolism in a subject of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than that of a non-encapsulated cannabinoid composition. Compositions in microencapsulated form can be characterized by a cannabinoid excretion rate from a subject of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than that of a non-encapsulated cannabinoid composition. Alternatively or in addition to, compositions in microencapsulated form can be characterized by a first pass non-cannabinoid compound metabolism in a subject of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than that of a non-encapsulated non-cannabinoid compound composition. Compositions in microencapsulated form can be characterized by a cannabinoid excretion rate from a subject of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than that of a non-encapsulated non-cannabinoid compound composition.

The compositions described herein, when administered to a subject, can have improved bioavailability, bioactivity, or both. Bioavailability is the fraction of an administered dosage of unchanged compound that reaches systemic circulation. Cannabinoid compositions in microencapsulated form can be characterized by a bioavailability in a subject of at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, or 10.0 times that of a non-encapsulated cannabinoid composition. Cannabinoid compositions in microencapsulated form can be characterized by a bioavailability in a subject of at least 5%, 10%, 15%, 20%, 25%, 30%, 35%, 40%, 45%, 50%, 55%, 60%, 65%, 70%, 75%, 80%, 85%, 90%, 95%, 96%, 97%,%, 98%, 99%, or 100%. Bioactivity, or biological activity, is the activity exerted by the active ingredient or ingredients in a composition. Cannabinoid compositions in microencapsulated form can be characterized by a bioactivity in a subject of at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, or 10.0 times that of a non-encapsulated cannabinoid composition. Alternatively or in addition to, non-cannabinoid compound compositions in microencapsulated form can be characterized by a bioavailability in a subject of at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, or 10.0 times that of a non-encapsulated non-cannabinoid compound composition. Non-cannabinoid compound compositions in microencapsulated form can be characterized by a bioavailability in a subject of at least 5%, 10%, 15%, 20%, 25%, 30%, 35%, 40%, 45%, 50%, 55%, 60%, 65%, 70%, 75%, 80%, 85%, 90%, 95%, 96%, 97%,%, 98%, 99%, or 100%. Bioactivity, or biological activity, is the activity exerted by the active ingredient or ingredients in a composition. Non-cannabinoid compound compositions in microencapsulated form can be characterized by a bioactivity in a subject of at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, or 10.0 times that of a non-encapsulated non-cannabinoid compound composition.

Subjects of the present disclosure can include humans and other animals, such as pets (e.g., dogs, cats, birds, small mammals, snakes) and livestock or farm animals (e.g., cows, pigs, horses, sheep, chickens). Compositions of the present disclosure can be useful for veterinary applications.

Methods and systems of the present disclosure may be used for forming compositions for various uses, such as encapsulating compositions (e.g., therapeutics compositions). Examples of uses of methods of the present disclosure are provided in WO/2016/094810, which is entirely incorporated herein by reference.

### EXAMPLES

### Example 1—Microencapsulation of Cannabinoids

A hemp oil composition is produced, comprising cannabinoids including cannabidiol. Additional essential oils are added to the composition. Alginate (e.g., sodium alginate) and quillaja tree extract are added to the composition. The composition is microencapsulated via a microfluidic nozzle device. Calcium chloride is used to cross-link the microcapsules. The microcapsules are packaged in a suspension, transported, and sold.

### Example 2—Administration of Cannabinoid Composition to a Subject

A cannabinoid composition, such as the microencapsulated cannabinoid composition described in Example 1, is administered to a subject suffering from a cannabinoid deficiency related condition. The level of cannabinoids in the subject increases, and the condition is improved.

### Example 3—Cannabidiol-Rich Hemp and Coconut Oil Product

Hempseed oil is enriched in cannabidiol compounds by addition of hemp stalk and stem extract containing 10% to 40% cannabidiol compounds by weight. The enriched hempseed oil is blended into coconut oil to produce a final composition of about 100 milligrams of cannabidiol compounds in 8 fluid ounces of coconut oil. The coconut oil product is then used to produce consumer products such as moisturizers, lotions, cooking oils, smoothies, spreads, and other food products.

### Example 4—Cannabidiolic Acid-Rich Hemp and Coconut Oil Product

Hempseed oil is enriched in cannabidiolic acid by addition of hemp stalk and stem extract containing 10% to 40% cannabidiolic acid by weight. The enriched hempseed oil is blended into coconut oil to produce a final composition of about 100 milligrams of cannabidiolic acid in 8 fluid ounces of coconut oil. The coconut oil product is then used to produce consumer products such as moisturizers, lotions, cooking oils, smoothies, spreads, and other food products.

### Example 5—Cannabidiol-Rich Hot Chocolate Mix

Hempseed oil is enriched in cannabidiol compounds by addition of hemp stalk and stem extract containing 10% to 40% cannabidiol compounds. Hempseed oil rich in cannabidiol compounds is then combined with cyclodextrin (e.g., certified organic cyclodextrin) to form a dry powder. The hemp oil powder is mixed with powdered cacao, cacao butter mix, sweeteners, and optionally superfood products such as reishi mushoom powder, chaga mushroom powder, maca, or he shou wu. The mixture is packaged and sold as a chocolate beverage mix (e.g., hot chocolate mix).

### Example 6—Production and Packaging of Cannabinoid-Rich Product

A standardized supercritical carbon dioxide extract of hemp stalk and stem is extracted. The extract (e.g., a paste) is blended into hemp seed oil. The blend of hemp extract and hemp oil is prepared with a THC content below 0.3%, and with CBD content of about 10-40% by weight. The hemp extract and hemp oil blend is further blended into coconut oil to provide about 100 mg of CBD per 8 ounce of coconut oil (about 423 milligrams per liter). The coconut oil blend with CBD is packaged (e.g., in a jar) and sold to a consumer.

### Example 7—Administration of Pregnenolone Composition to a Subject

A cannabinoid and pregnenolone composition, such as the microencapsulated cannabinoid composition described in Example 1 further comprising pregnenolone (e.g., 1-50 mg of pregnenolone), is administered to a subject suffering from cannabinoid intoxication or addiction. The subject is protected from CB1 receptor overactivation, and the condition is improved.

### Example 8—Preparation of Microencapsulated Composition

Formulations comprising quillaja extract (e.g., Q Natural), hemp oil, water, and optionally sodium alginate were prepared using a microfluidic fluid processor (e.g., Microfluidizer from Microfluidics/IDEX Corporation). Formulations were prepared as described in Table 1. Test 1 was prepared with 60 g of quillaja extract (e.g., Q Natural), 80 g of hemp oil, and 100 g of water, at an operating pressure of 30,000 psi in the microfluidic fluid processor. Test 2 was prepared with 10 g of quillaja extract (e.g., Q Natural), 15 g of hemp oil, 198 g of water, and 2 g of sodium alginate, at an operating pressure of 30,000 psi in the microfluidic fluid processor.

After processing in the microfluidic fluid processor, particle size distribution was analyzed using a laser diffraction particle size analyzer (e.g., Horiba LA950). Optical microscope images were also taken.

Three passes each of Test 1 and Test 2 were conducted, and the tenth percentile (D10), fiftieth percentile (D50), and ninetieth percentile (D90) particle sizes are reported in Table 1. Particle sizes were also analyzed for an unprocessed solution (Test 1, Pass 0).

TABLE 1
Formulation and size distribution information for microencapsulated compositions.

| | Pass | | Q | Hemp | | Sodium | D10 | D50 | D90 |
|---|---|---|---|---|---|---|---|---|---|
| Test | # | Pressure | Natural | Oil | Water | alginate | (μm) | (μm) | (μm) |
| 1 | 0 | 30,000 psi | 60 g | 80 g | 100 g | — | 1.9737 | 7.117 | 35.703 |
| | 1 | | | | | — | 0.0768 | 0.1296 | 0.2881 |
| | 2 | | | | | — | 0.0697 | 0.1096 | 0.1791 |
| | 3 | | | | | — | 0.0929 | 0.1405 | 0.2202 |
| 2 | 1 | 30,000 psi | 10 g | 15 g | 198 g | 2 g | 0.1171 | 0.1965 | 2.0719 |
| | 2 | | | | | | 0.0688 | 0.1097 | 0.2209 |
| | 3 | | | | | | 0.099 | 0.1583 | 1.3443 |

FIG. 1A shows a 400× magnification micrograph image of an unprocessed quillaja extract, hemp oil, and water composition (Test 1, Pass 0), with a 50 μm scale bar. FIG. 1B shows a 1000× magnification micrograph image of an unprocessed quillaja extract, hemp oil, and water composition (Test 1, Pass 0), with a 50 μm scale bar.

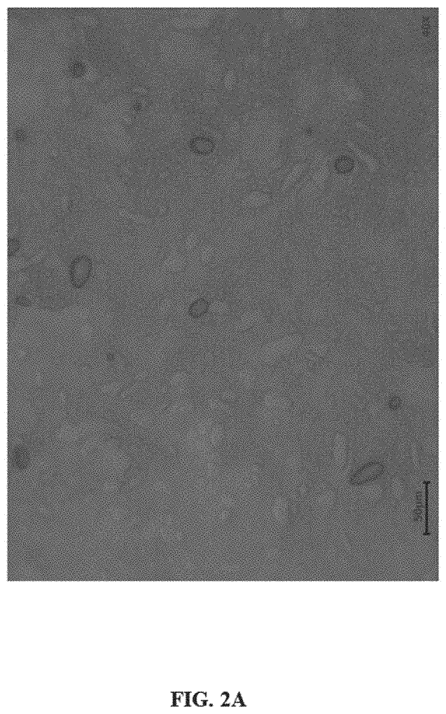

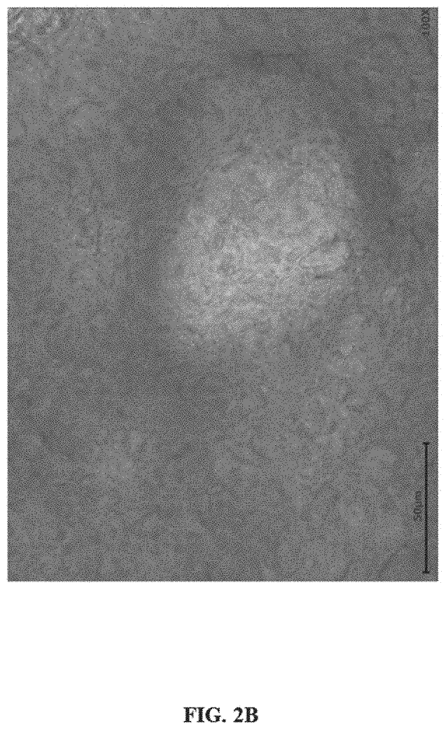

FIG. 2A shows a 400× magnification micrograph image of a quillaja extract, hemp oil, and water composition (Test 1, Pass 1), with a 50 μm scale bar. FIG. 2B shows a 400× magnification micrograph image of a quillaj a extract, hemp oil, and water composition (Test 1, Pass 2), with a 50 μm scale bar. FIG. 2C shows a 1000× magnification micrograph image of a quillaja extract, hemp oil, and water composition (Test 1, Pass 2), with a 10 μm scale bar. FIG. 2D shows a 1000× magnification micrograph image of a quillaja extract, hemp oil, and water composition (Test 1, Pass 3), with a 10 μm scale bar.

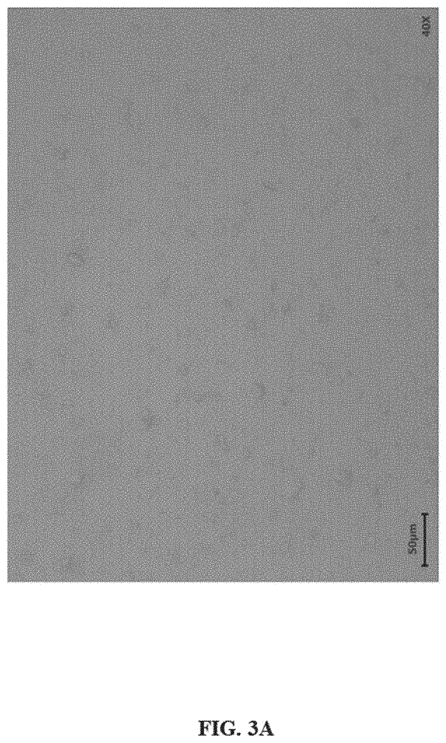

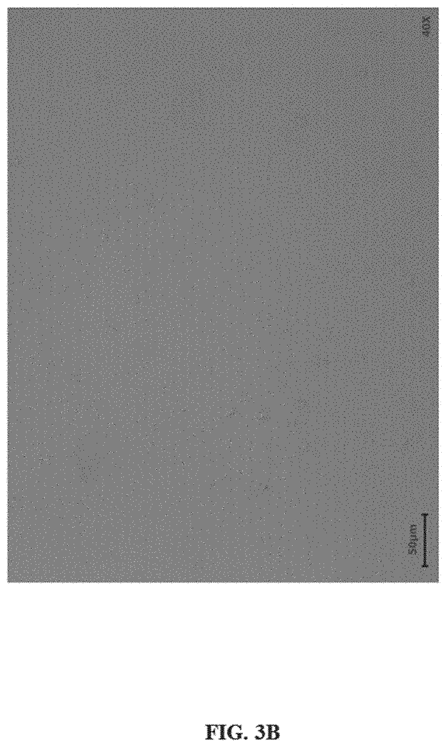

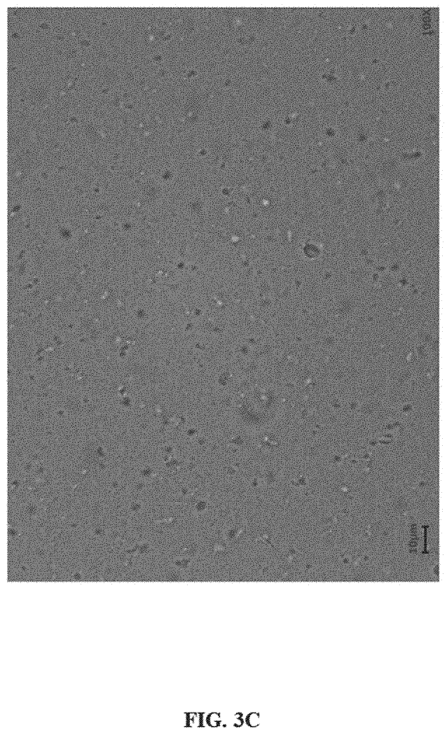

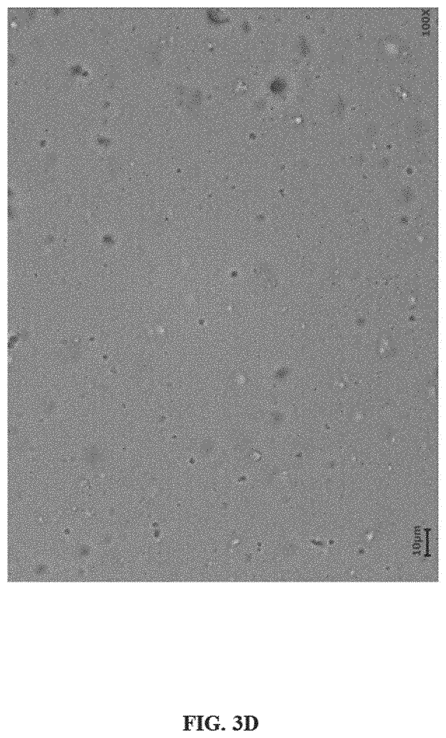

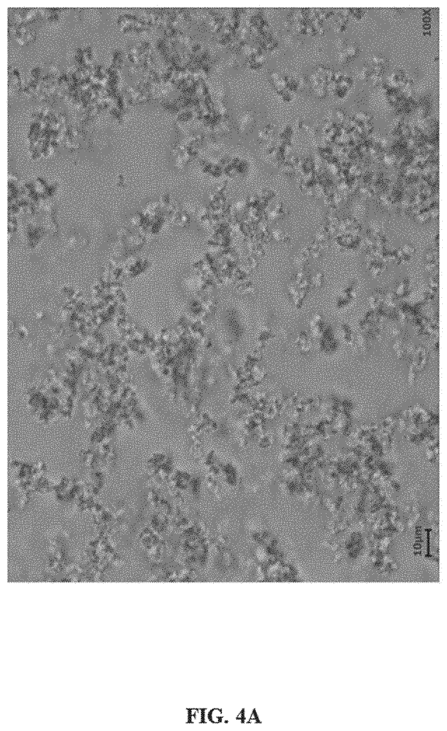

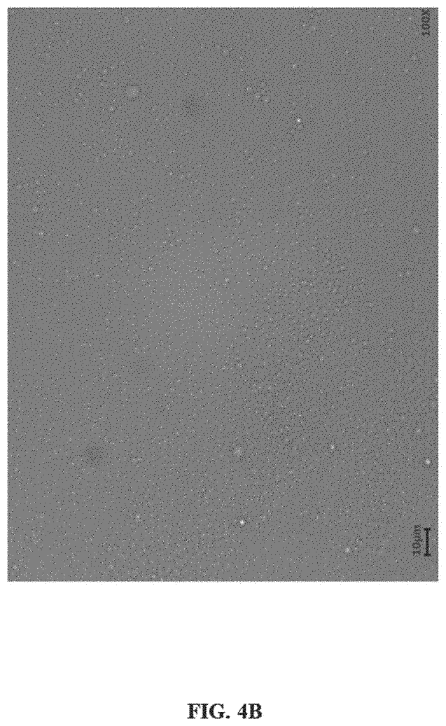

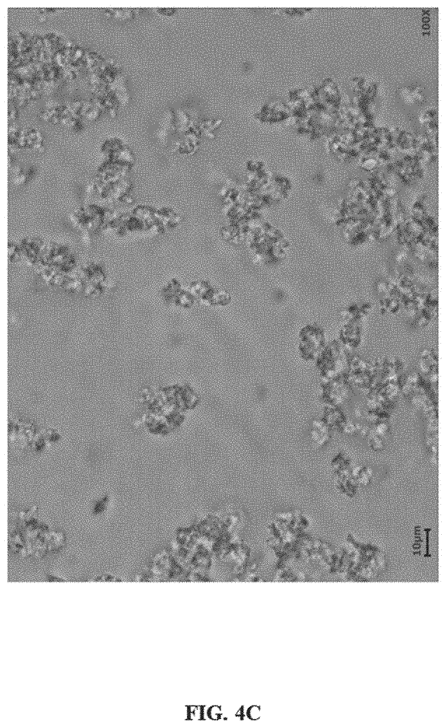

FIG. 3A shows a 1000× magnification micrograph image of a quillaja extract, hemp oil, water, and sodium alginate composition (Test 2, Pass 1), with a 10 μm scale bar. FIG. 3B shows a 1000× magnification micrograph image of a quillaja extract, hemp oil, water, and sodium alginate composition (Test 2, Pass 2), with a 10 μm scale bar. FIG. 3C shows a 1000× magnification micrograph image of a quillaj a extract, hemp oil, water, and sodium alginate composition (Test 2, Pass 3), with a 10 μm scale bar.

### Example 9—Therapeutic Efficacy of Compositions Comprising Cannabinoid(s)

A therapeutic composition as disclosed herein can be administered to a subject having or is suspected of having a condition, such as a viral infection (e.g., influenza, coronavirus infection, etc.). Table 2 shows examples of therapeutic compositions comprising (i) cannabinoids (e.g., encapsulated or non-encapsulated cannabinoids, such as cannabidiol) and/or (ii) additional non-cannabinoid therapeutic compounds, where each therapeutic composition (as indicated by the sample number) is for being administered (e.g., orally) to a subject in need thereof.

TABLE 2
Compositions comprising encapsulated cannabinoids.

| | | Additional therapeutic | | Are CC and ATC |
|---|---|---|---|---|
| | Cannabinoid | compound (ATC) | | encapsulated |
| | Compound | | Encapsu- | in the same |
| No. | (CC) | Type | lation | microcapsule? |
| 1 | Encapsulated | No | n/a | n/a |
| 2 | Encapsulated | ACE inhibitor | No | n/a |
| 3 | Encapsulated | ACE inhibitor | Yes | Yes |
| 4 | Encapsulated | ACE inhibitor | Yes | No |
| 5 | Encapsulated | Angiotensin | No | n/a |
| | | receptor II blocker | | |
| 6 | Encapsulated | Angiotensin | Yes | Yes |
| | | receptor II blocker | | |
| 7 | Encapsulated | Angiotensin | Yes | No |
| | | receptor II blocker | | |
| 8 | Non- | No | n/a | n/a |
| | encapsulated | | | |

(“n/a” = not applicable).

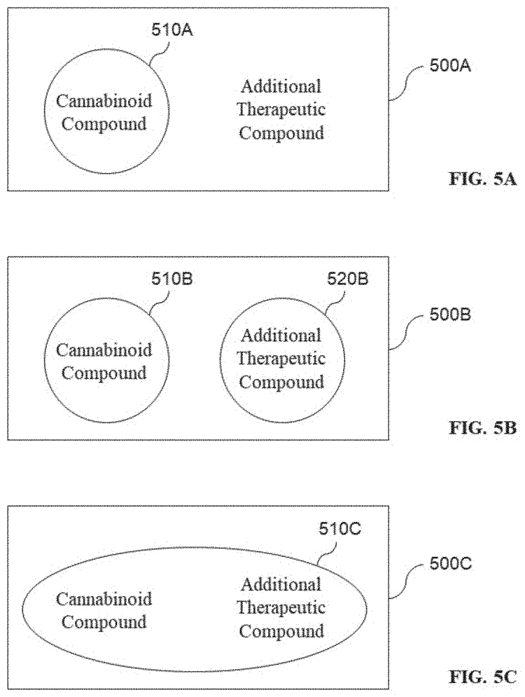

FIG. 5A shows an example composition 500A (e.g., composition 2 or composition 5 from Table 2) comprising one or more microcapsules 510A that encapsulate one or more cannabinoid compounds. The composition 500A further comprises one or more additional therapeutic compounds (e.g., ACE inhibitor, angiotensin receptor II blocker) that is not encapsulated within microcapsules.

FIG. 5B shows an example composition 500B (e.g., composition 4 or composition 7 from Table 2) comprising one or more microcapsules 510B that encapsulate one or more cannabinoid compounds. The composition 500B further comprises one or more additional microcapsules 520B that encapsulate one or more additional therapeutic compounds (e.g., ACE inhibitor, angiotensin receptor II blocker).

FIG. 5C shows an example composition 500C (e.g., composition 3 or composition 6 from Table 2) comprising one or more microcapsules 510C that encapsulate both (i) one or more cannabinoid compounds and (ii) one or more additional therapeutic compounds (e.g., ACE inhibitor, angiotensin receptor II blocker).

A subject can be administered with (e.g., via oral administration) a selected composition from Table 2. The subject can be administered with one dose or multiple doses (e.g., at least or up to about 1, 2, 3, 4, 5, 6, 7, 8, 9, 10 doses) of the composition. The dose(s) can be administered to the subject within a therapeutic window, such as a predetermined time period (e.g., at least or up to about 1 day, 2 days, 3 days, 4 days, 5 days, 6 days, 7 days, 2 weeks, 3 weeks, 4 weeks, 2 months, 3 months, or 4 months). During or subsequent to the therapeutic window, various parameters of the subject can be measured (e.g., via analyzing one or more biological samples from the subject, such as a blood sample from the subject) to ascertain effects of administering the composition to the subject, as compared to a control subject (e.g., a subject having been administered with a placebo, such as empty microcapsules, or with a different composition from Table 2, such as composition number 8 from Table 2).

A subject administered with any one of the compositions 2-7 from Table 2 can exhibit (i) reduced expression or activity of a cytokine in a target cell, when the administering occurs to the subject having a viral infection, (ii) reduced platelet activation, and/or (iii) reduced expression or activity of angiotensin pathway protein in a target cell, as compared to (1) that of the subject prior to the administration or (2) a control subject who is administered with composition 1 or composition 8 from Table 2.

A subject administered with composition 3 or composition 4 from Table 2 can exhibit (i) reduced expression or activity of a cytokine in a target cell, when the administering occurs to the subject having a viral infection, (ii) reduced platelet activation, and/or (iii) reduced expression or activity of angiotensin pathway protein in a target cell, as compared to a control subject who is administered with composition 2 from Table 2.

A subject administered with composition 4 from Table 2 can exhibit (i) reduced expression or activity of a cytokine in a target cell, when the administering occurs to the subject having a viral infection, (ii) reduced platelet activation, and/or (iii) reduced expression or activity of angiotensin pathway protein in a target cell, as compared to a control subject who is administered with composition 3 from Table 2. Alternatively, a subject administered with composition 3 from Table 2 can exhibit (i) reduced expression or activity of a cytokine in a target cell, when the administering occurs to the subject having a viral infection, (ii) reduced platelet activation, and/or (iii) reduced expression or activity of angiotensin pathway protein in a target cell, as compared to a control subject who is administered with composition 4 from Table 2.

A subject administered with composition 6 or composition 7 from Table 2 can exhibit (i) reduced expression or activity of a cytokine in a target cell, when the administering occurs to the subject having a viral infection, (ii) reduced platelet activation, and/or (iii) reduced expression or activity of angiotensin pathway protein in a target cell, as compared to a control subject who is administered with composition 5 from Table 2.

A subject administered with composition 7 from Table 2 can exhibit (i) reduced expression or activity of a cytokine in a target cell, when the administering occurs to the subject having a viral infection, (ii) reduced platelet activation, and/or (iii) reduced expression or activity of angiotensin pathway protein in a target cell, as compared to a control subject who is administered with composition 6 from Table 2. Alternatively, a subject administered with composition 6 from Table 2 can exhibit (i) reduced expression or activity of a cytokine in a target cell, when the administering occurs to the subject having a viral infection, (ii) reduced platelet activation, and/or (iii) reduced expression or activity of angiotensin pathway protein in a target cell, as compared to a control subject who is administered with composition 7 from Table 2.

1. A method for reducing expression of activity of a cytokine in a target cell comprising administering to a subject in need thereof a therapeutically effective amount of a composition comprising a plurality of microcapsules, wherein a microcapsule of the plurality comprises at least one cannabinoid compound,
wherein the administering effects, in the subject, one or more members selected from the group consisting of (i) reduced expression or activity of a cytokine in a target cell, when the administering occurs to the subject having a viral infection, (ii) reduced platelet activation, and (iii) reduced expression or activity of angiotensin pathway protein in a target cell, as compared to a corresponding control.

2. The method of claim 1, wherein the administering effects the reduced expression or activity of the cytokine in the target cell.

3. The method of claim 1 or 2, wherein the administering effects at least about 20% reduction in the expression or activity of (i) the cytokine and (ii) an additional cytokine in the target cell.

4. The method of any one of the preceding claims, wherein the cytokine or the additional cytokine is selected from the group consisting of an interferon, an interleukin, a chemokine, a colony-stimulating factor, and a tumor necrosis factor.

5. The method of any one of the preceding claims, wherein the administering effects reduced cytokine storm in the subject.

6. The method of claim 1, wherein the administering effects the reduced platelet activation.

7. The method of claim 6, wherein the administering effects at least about 20% reduction in expression level of a chemokine in the platelet, wherein the chemokine is selected from the group consisting of CXCL1, CXCL4, CXCL5, CXCL7, CXCL8, CXCL12, CCL3, and CCL5.

8. The method of claim 6, wherein the administering effects at least about 20% reduction in expression level of a cell-adhesion molecule in the platelet, wherein the cell-adhesion molecule is selected from the group consisting of glycoprotein IIb/IIIa, P-selectin, and integrin.

9. The method of claim 6, wherein the administering effects at least about 20% reduction in expression level of adenosine diphosphate (ADP) or adenosine triphosphate (ATP) in the platelet.

10. The method of any one of claims 6-9, wherein the administering effects reduced blood clot formation in (i) a blood vessel of the subject or (ii) a blood sample derived from the subject.

11. The method of claim 10, wherein, upon the administering, the blood sample exhibits at least about 20% reduction in platelet aggregation as measured by light-transmission aggregometry (LTA) assay or whole blood aggregometry (WBA) assay.

12. The method of claim 1, wherein the administering effects the reduced expression or activity of the angiotensin pathway protein in the target cell.

13. The method of claim 12, wherein the administering effects at least about 20% reduction in the expression or activity of the angiotensin pathway protein.

14. The method of claim 12 or 13, wherein the angiotensin pathway protein is selected from the group consisting of angiotensin converting enzyme (ACE) and angiotensin receptor.

15. The method of claim 12 or 13, wherein the angiotensin pathway protein is selected from the group consisting of ACE2 and angiotensin II receptor.

16. The method of any one of the preceding claims, wherein the composition further comprises an additional therapeutic compound is selected from the group consisting of hydroxychloroquine, chroloquine, azithromycin, dexamethasone, steroids, vitamins C, vitamin D, stem cells, baloxavir, chloroquine phosphate, lopinavir, ritonavir, neuraminidase inhibitors, remedesivir, ascorbic acid, methylprednisolone, nitric oxide, sarilumab, sirolimus, tocilizumab, ACE inhibitors, angiotensin II receptor blockers (ARBs), ibuprofen, indomethacin, and niclosamide.

17. The method of claim 16, wherein the additional therapeutic compound is encapsulated in a microcapsule of the plurality of microcapsules.

18. The method of claim 17, wherein the at least one cannabinoid compound and the additional therapeutic compound are encapsulated in the same microcapsule.

19. The method of claim 17, wherein the at least one cannabinoid compound and the additional therapeutic compound are encapsulated in different microcapsules.

20. The method of claim 16, wherein the additional therapeutic compound is provided in non-encapsulated form.

21.-30. (canceled)
